SEQUENCE LISTING (13 sequences)

SEQ ID NO: 1 - DNA, 2481 bp, Homo sapiens
  CDS (1)..(2481)
atggcggagccctcggccccggagagcaagcacaagtcgtccctcaactcgtccccgtggagtggcctcatggccctgggaaacagccggcacggccaccacgggcccggggcccagtgcgcgcacaaggcggcgggcggcgcggcgccgccgaagccggccccggcggggctgtccggggggctgtcgcagccggctgggtggcagtcgcttctctccttcaccatcctcttcctggcctggcttgccggcttcagctcgcgcctcttcgccgtcatccgcttcgaaagcatcatccacgagttcgacccgtggtttaactatagatcaacacatcatcttgcatctcatgggttctatgaatttttaaattggtttgatgaaagagcatggtatccactaggaagaatagtaggtggtactgtttacccagggttgatgataaccgctggccttattcattggattttaaatacattgaacataactgttcacataagagacgtatgtgtgttccttgcaccaacttttagcggccttacatctatatctactttcctgcttacaagagaactttggaaccaaggagcaggacttttagctgcttgttttattgctattgtaccaggctacatatctcggtcagtagctggatcctttgataatgaaggcattgctatttttgcacttcagttcacatactatttatgggtaaaatctgtaaaaactgggtcagttttttggacaatgtgctgctgcttatcctatttctatatggtctctgcttggggtggttatgtatttatcatcaatcttattccactgcatgtatttgtgttgttactgatgcagagatacagcaaaagagtctacatagcatatagcactttctacattgtgggtttaatattatcaatgcagataccttttgtgggattccagccaatcagaacaagtgaacacatggcagctgcaggtgtctttgcattgctgcaagcttatgctttcttgcagtatctgagagaccgattaacaaaacaagagttccagacccttttctttttgggtgtatcactagctgcaggtgctgtgttccttagtgtcatctatttgacttatacaggttacattgcaccatggagtggcaggttttattcattgtgggatactgggtatgcaaaaatacacattccaattattgcatcagtgtctgagcatcaacctacgacttgggtgtctttcttctttgatctacatattcttgtatgtaccttcccagcaggcctttggttctgcatcaaaaatatcaacgatgaaagagtatttgttgctctatatgcaatcagtgctgtctactttgctggagtgatggtgcgactgatgttgactttgactccagtcgtgtgtatgctgtctgcaattgccttttcaaatgtttttgagcactatttgggggatgacatgaaaagggaaaatccacctgtggaggacagcagtgatgaggatgacaaaagaaaccaaggaaatttgtatgataaggcaggtaaagtgaggaaacatgcaactgaacaggaaaaaactgaagagggattaggccctaatataaaaagcattgtcaccatgttgatgctgatgctattgatgatgtttgctgtccactgtacctgggtcacaagcaatgcctactctagtccaagtgtagtcctggcctcatacaatcatgatggcaccaggaatatcttagatgattttagagaagcttacttttggctaaggcaaaatacagatgaacatgcacgagtaatgtcttggtgggattatggctatcagatagctggaatggctaatagaactacgttggtggataataacacctggaataacagccacatagcactggtgggaaaagctatgtcttctaatgaaacagcagcctataaaatcatgaggactctagatgtagattatgttttggttatttttggaggggttattggctattctggtgatgatatcaacaaatttctctggatggttaggatagctgaaggagaacatcccaaagacattcgggaaagtgactattttaccccacagggagaattccgtgtagacaaagcaggatcccctactttgttgaattgccttatgtataaaatgtcatactacagatttggagaaatgcagctggattttcgtacacccccaggttttgaccgaacacgtaatgctgagattggaaataaggacattaaattcaaacatttggaagaagcctttacatcagaacactggcttgttaggatatataaagtaaaagcacctgataacagggagacattagatcacaaacctcgagtcaccaacattttcccaaaacagaagtatttgtcaaagaagactaccaaaaggaagcgtggctacattaaaaataagctggtttttaagaaaggcaagaaaatatctaagaagactgtttaa

SEQ ID NO: 2 - protein, 826 aa, Homo sapiens
MAEPSAPESKHKSSLNSSPWSGLMALGNSRHGHHGPGAQCAHKAAGGAAPPKPAPAGLSGGLSQPAGWQSLLSFTILFLAWLAGFSSRLFAVIRFESIIHEFDPWFNYRSTHHLASHGFYEFLNWFDERAWYPLGRIVGGTVYPGLMITAGLIHWILNTLNITVHIRDVCVFLAPTFSGLTSISTFLLTRELWNQGAGLLAACFIAIVPGYISRSVAGSFDNEGIAIFALQFTYYLWVKSVKTGSVFWTMCCCLSYFYMVSAWGGYVFIINLIPLHVFVLLLMQRYSKRVYIAYSTFYIVGLILSMQIPFVGFQPIRTSEHMAAAGVFALLQAYAFLQYLRDRLTKQEFQTLFFLGVSLAAGAVFLSVIYLTYTGYIAPWSGRFYSLWDTGYAKIHIPIIASVSEHQPTTWVSFFFDLHILVCTFPAGLWFCIKNINDERVFVALYAISAVYFAGVMVRLMLTLTPVVCMLSAIAFSNVFEHYLGDDMKRENPPVEDSSDEDDKRNQGNLYDKAGKVRKHATEQEKTEEGLGPNIKSIVTMLMLMLLMMFAVHCTWVTSNAYSSPSVVLASYNHDGTRNILDDFREAYFWLRQNTDEHARVMSWWDYGYQIAGMANRTTLVDNNTWNNSHIALVGKAMSSNETAAYKIMRTLDVDYVLVIFGGVIGYSGDDINKFLWMVRIAEGEHPKDIRESDYFTPQGEFRVDKAGSPTLLNCLMYKMSYYRFGEMQLDFRTPPGFDRTRNAEIGNKDIKFKHLEEAFTSEHWLVRIYKVKAPDNRETLDHKPRVTNIFPKQKYLSKKTTKRKRGYIKNKLVFKKGKKISKKTV

SEQ ID NO: 3 - DNA, 2710 bp, Mus musculus
  ref <308>: DATABASE ACCESSION NUMBER: AK018758
  ref <309>: DATABASE ENTRY DATE: 2001-07-05
  ref <313>: RELEVANT RESIDUES: (1)..(2469)
cgccgcccagcacccctcgctccaggcggcggcggtggccgcggaggacgagcgagacccgccgccggggcacaacatggcggagccctcggccccggagagcaagcacaagtcgtccctcaactcgtccccgtggagcggcctcatggctctggggaacagccgccacgggcaccatgggcccggaacccagagcgcgtccagggcggcggcgccgaagccggggccccccgcggggctgtccgggggcttgtcgcagccggccgggtggcagtcgttgctctccttcaccatcctcttcctggcctggctggccggcttcagctcgcgcctcttcgccgtcatccgcttcgagagcatcatccacgagttcgacccgtggtttaactatagatcaacacatcatcttgcatctcatggattctatgagtttctaaattggtttgatgaaagagcatggtacccactgggaagaatagtgggtggcaccgtttacccagggttgatgataacagctggccttattcattggattttaaatacattgaacataacagttcacataagagatgtgtgtgtattccttgcaccaacttttagcggccttacatccatatctacgttcctgctaactagagaactgtggaaccaaggagcaggacttctagctgcttgcttcattgctatcgtaccagggtacatatctcggtcagtggcgggatcctttgataatgaaggcattgccatttttgcgcttcagttcacttactacttatgggtaaagtctgtgaagaccgggtctgtgttctggacaatgtgctgctgcttgtcatatttctacatggtctctgcgtggggaggttatgtgttcatcatcaacctcatccctctccatgtgtttgtgttgctgctgatgcagaggtacagcaagagagtctacatagcatatagcactttgtacattgtgggtttaatattatccatgcagataccttttgtgggatttcagccaatcagaacaagcgagcacatggcagctgcaggtgtctttgcgctgctgcaagcttacgcttttttgcagtatctgagagaccggttgacaaaacaggagttccagacccttttctttttgggtgtctcactagctgcaggcgctgtgttccttagtgtcatctatctgacatacacaggttatattgcaccatggagtggcaggttttattcactatgggatactgggtatgcaaaaatacacattccaattattgcatcagtgtctgaacatcagcctacgacatgggtgtctttcttctttgatctacatattcttgtatgtaccttcccagcaggcctatggttctgcatcaaaaatatcaacgatgaaagagtatttgtcgctctgtatgcgatcagtgctgtgtactttgccggagtgatggtgcggctgatgctgactctgaccccggtcgtctgcatgctgtcggccatcgccttctccaatgtttttgagcactatttgggggatgacatgaaaagggaaaacccacctgtggaggacagcagtgatgaggatgacaaaagaaacccaggaaacttgtatgacaaggcaggtaaagtgaggaagcatgtgacagagcaagagaaacctgaagagggcttgggccccaacatcaaaagcattgtgaccatgctgatgctcatgctcctgatgatgttcgcggtccactgcacgtgggtcacaagcaacgcctactccagtccaagtgtggtccttgcctcctacaatcatgatggtaccaggaatatattagatgattttagagaagcgtacttttggctgagacaaaacacggatgaacacgcccgggtcatgtcgtggtgggactacggctatcagattgctggcatggccaacaggaccactctggtggataacaacacctggaacaacagccacatcgcactggtcggaaaagctatgtcttccaatgaaacggccgcctataaaatcatgaggtcccttgatgtcgattatgtgttggttattttcggaggagtgattggctattccggggacgatatcaacaagttcctctggatggtcaggatagctgaaggggagcatcccaaagacatccgggaaggtgactatttcacccagcagggagagttccgagtagacaaagctgggtctcctactctgttaaactgccttatgtataaaatgtcatactacagatttggagaaatgcagctagattttcgcactcccccaggctttgaccgaacacgtaatgctgagattggaaataaagacattaaattcaagcatttggaggaagcttttacatcagagcactggcttgtcaggatatataaagtgaaagcacctgacaacagggagacactaggtcacaaacctcgagtcaccaacatcgtccccaaacagaagtatttgtcaaagaagactactaaaaggaagcgtggctacgttaaaaataagctagtgtttaagaaaggcaagaagacctctaagaagactgtttaaatgcgctgttctggcctcacttgcagcagtccttgagagaaccggtctttgccttctgctcatgtcctgtttcacagcaccaagggtacagaacatcgctgggccaagtcaatgtacaaaatgttctggcaatgcctcatttaaaattaaattggtttattg

SEQ ID NO: 4 - protein, 823 aa, Mus musculus
  ref <308>: DATABASE ACCESSION NUMBER: AK018758
  ref <309>: DATABASE ENTRY DATE: 2001-07-05
  ref <313>: RELEVANT RESIDUES: (1)..(823)
MAEPSAPESKHKSSLNSSPWSGLMALGNSRHGHHGPGTQSASRAAAPKPGPPAGLSGGLSQPAGWQSLLSFTILFLAWLAGFSSRLFAVIRFESIIHEFDPWFNYRSTHHLASHGFYEFLNWFDERAWYPLGRIVGGTVYPGLMITAGLIHWILNTLNITVHIRDVCVFLAPTFSGLTSISTFLLTRELWNQGAGLLAACFIAIVPGYISRSVAGSFDNEGIAIFALQFTYYLWVKSVKTGSVFWTMCCCLSYFYMVSAWGGYVFIINLIPLHVFVLLLMQRYSKRVYIAYSTLYIVGLILSMQIPFVGFQPIRTSEHMAAAGVFALLQAYAFLQYLRDRLTKQEFQTLFFLGVSLAAGAVFLSVIYLTYTGYIAPWSGRFYSLWDTGYAKIHIPIIASVSEHQPTTWVSFFFDLHILVCTFPAGLWFCIKNINDERVFVALYAISAVYFAGVMVRLMLTLTPVVCMLSAIAFSNVFEHYLGDDMKRENPPVEDSSDEDDKRNPGNLYDKAGKVRKHVTEQEKPEEGLGPNIKSIVTMLMLMLLMMFAVHCTWVTSNAYSSPSVVLASYNHDGTRNILDDFREAYFWLRQNTDEHARVMSWWDYGYQIAGMANRTTLVDNNTWNNSHIALVGKAMSSNETAAYKIMRSLDVDYVLVIFGGVIGYSGDDINKFLWMVRIAEGEHPKDIREGDYFTQQGEFRVDKAGSPTLLNCLMYKMSYYRFGEMQLDFRTPPGFDRTRNAEIGNKDIKFKHLEEAFTSEHWLVRIYKVKAPDNRETLGHKPRVTNIVPKQKYLSKKTTKRKRGYVKNKLVFKKGKKTSKKTV

SEQ ID NO: 5 - DNA, 2733 bp, Saccharomyces cerevisiae
  ref <308>: DATABASE ACCESSION NUMBER: D28952
  ref <309>: DATABASE ENTRY DATE: 1999-02-07
  ref <313>: RELEVANT RESIDUES: (1)..(2733)
aagctttcttttacttctcttcgcctctgctaaatggtcaccatcgacggttgctttttcgcgctggtcgagaattgacaaaataagacacgaacaaaagagcaagtctgaaagaaagaaaagcagcaaaagcacggtctaattcaacgtgacatagcatccgcaatcgcattcacagccgtaaatcctaactaccattcgtcattatcacagctgccatgggatccgaccggtcgtgtgttttgtctgtgtttcagaccatcctcaagctcgtcatcttcgtggcgatttttggggctgccatatcatcacgtttgtttgcagtcatcaaatttgagtctattatccatgaattcgacccctggttcaattatagggctaccaaatatctcgtcaacaattcgttttacaagtttttgaactggtttgacgaccgtacctggtaccccctcggaagggttactggagggactttatatcctggtttgatgacgactagtgcgttcatctggcacgccctgcgcaactggttgggcttgcccattgacatcagaaacgtttgtgtgctatttgcgccactattttctggggtcaccgcctgggcgacttacgaatttacgaaagagattaaagatgccagcgctgggcttttggctgctggttttatagccattgtccccggttatatatctagatcagtggcggggtcctacgataatgaggccattgccattacactattaatggtcactttcatgttttggattaaggcccaaaagactggctctatcatgcacgcaacgtgtgcagctttattctacttctacatggtgtcggcttggggtggatacgtgttcatcaccaacttgatcccactccatgtctttttgctgattttgatgggcagatattcgtccaaactgtattctgcctacaccacttggtacgctattggaactgttgcatccatgcagatcccatttgtcggtttcctacctatcaggtctaacgaccacatggccgcattgggtgttttcggtttgattcagattgtcgccttcggtgacttcgtgaagggccaaatcagcacagctaagtttaaagtcatcatgatggtttctctgtttttgatcttggtccttggtgtggtcggactttctgccttgacctatatggggttgattgccccttggactggtagattttattcgttatgggataccaactacgcaaagatccacattcctatcattgcctccgtttccgaacatcaacccgtttcgtggcccgctttcttctttgatacccactttttgatctggctattccccgccggtgtattcctactattcctcgacttgaaagacgagcacgtttttgtcatcgcttactccgttctgtgttcgtactttgccggtgttatggttagattgatgttgactttgacaccagtcatctgtgtgtccgccgccgtcgcattgtccaagatatttgacatctacctggatttcaagacaagtgaccgcaaatacgccatcaaacctgcggcactactggccaaattgattgtttccggatcattcatcttttatttgtatcttttcgtcttccattctacttgggtaacaagaactgcatactcttctccttctgttgttttgccatcacaaaccccagatggtaaattggcgttgatcgacgacttcagggaagcgtactattggttaagaatgaactctgatgaggacagtaaggttgcagcgtggtgggattacggttaccaaattggtggcatggcagacagaaccactttagtcgataacaacacgtggaacaatactcacatcgccatcgttggtaaagccatggcttcccctgaagagaaatcttacgaaattctaaaagagcatgatgtcgattatgtcttggtcatctttggtggtctaattgggtttggtggtgatgacatcaacaaattcttgtggatgatcagaattagcgagggaatctggccagaagagataaaagagcgttatttctataccgcagagggagaatacagagtagatgcaagggcttctgagaccatgaggaactcgctactttacaagatgtcctacaaagatttcccacaattattcaatggtggccaagccactgacagagtgcgtcaacaaatgatcacaccattagacgtcccaccattagactacttcgacgaagtttttacttccgaaaactggatggttagaatatatcaattgaagaaggatgatgcccaaggtagaactttgagggacgttggtgagttaaccaggtcttctacgaaaaccagaaggtccataaagagacctgaattaggcttgagagtctaaattggccacacattaaaggaaatgactaagataaaatatacatatataaaaagataaacaaataagtataagtttggtttcccttcccgttattatgatcgctcgtgacggatcgtctttgccctttttggtaaaacgtaaacaaaataacaatagaaaaaataacaactttatcaatgtttatttttatttattaagtatttgatgtgaagtagtttttctaaatgctacttcattttgacattgtaattcaattactatcaagtcatacccttaaatcgcaccaagtagagccccccatggattttgaaacgtcgttcgaagaatttgtcgaagataaacgattcattgctctaga

SEQ ID NO: 6 - protein, 718 aa, Saccharomyces cerevisiae
  ref <308>: DATABASE ACCESSION NUMBER: BAA06079
  ref <309>: DATABASE ENTRY DATE: 1999-02-07
  ref <313>: RELEVANT RESIDUES: (1)..(718)
MGSDRSCVLSVFQTILKLVIFVAIFGAAISSRLFAVIKFESIIHEFDPWFNYRATKYLVNNSFYKFLNWFDDRTWYPLGRVTGGTLYPGLMTTSAFIWHALRNWLGLPIDIRNVCVLFAPLFSGVTAWATYEFTKEIKDASAGLLAAGFIAIVPGYISRSVAGSYDNEAIAITLLMVTFMFWIKAQKTGSIMHATCAALFYFYMVSAWGGYVFITNLIPLHVFLLILMGRYSSKLYSAYTTWYAIGTVASMQIPFVGFLPIRSNDHMAALGVFGLIQIVAFGDFVKGQISTAKFKVIMMVSLFLILVLGVVGLSALTYMGLIAPWTGRFYSLWDTNYAKIHIPIIASVSEHQPVSWPAFFFDTHFLIWLFPAGVFLLFLDLKDEHVFVIAYSVLCSYFAGVMVRLMLTLTPVICVSAAVALSKIFDIYLDFKTSDRKYAIKPAALLAKLIVSGSFIFYLYLFVFHSTWVTRTAYSSPSVVLPSQTPDGKLALIDDFREAYYWLRMNSDEDSKVAAWWDYGYQIGGMADRTTLVDNNTWNNTHIAIVGKAMASPEEKSYEILKEHDVDYVLVIFGGLIGFGGDDINKFLWMIRISEGIWPEEIKERYFYTAEGEYRVDARASETMRNSLLYKMSYKDFPQLFNGGQATDRVRQQMITPLDVPPLDYFDEVFTSENWMVRIYQLKKDDAQGRTLRDVGELTRSSTKTRRSIKRPELGLRV

SEQ ID NO: 7 - DNA, 2417 bp, Drosophila melanogaster
  ref <308>: DATABASE ACCESSION NUMBER: AF132552
  ref <309>: DATABASE ENTRY DATE: 1999-04-27
  ref <313>: RELEVANT RESIDUES: (1)..(2417)
tctaagcgaagaatgtgtcgttgcatttcagatcggttataattttcgagttactggctggaattgggacatgaatcggacgccgaagatgctgaacagcaaggtggctggctacagcagcctaatcaccttcgccatcctgctaatcgcctggctggccggattttcctctcgcctcttcgccgtcatccgtttcgagtcgattatccatgagtttgatccgtggttcaactaccgggccaccgcctacatggtgcagaatggttggtacaacttcctcaactggttcgacgagcgcgcatggtatccgctcggcaggattgtgggcggtaccgtctatcccggcctgatgattacgtccggcggaatccattggctgctgcacgtactcaacataccggtccatattcgtgacatctgcgtgttcctggcgccgatcttcagtggcctgacctccatctccacctacctgctgaccaaggagctgtggtccgcgggcgccggcctcttcgccgccagcttcatcgccatcgtgcctggctacatcagtaggtcggtggctggatcgtacgataacgagggcattgccatattcgccctgcagttcacctacttcctgtgggtgcgctcagtgaagactggatccgtgttctggtcggccgcagccgctttgtcctacttctacatggtgtccgcctggggtggctacgtgttcatcatcaacctgatacccctgcacgtcttcgtactgctcattatgggcaggtactcgccgcgtctgctgaccagctacagcaccttctacatcctgggactgctgttctccatgcagatccccttcgtgggattccaaccgatacgcaccagtgaacacatggctgcgctgggagtgtttgtgctccttatggccgtggccaccttgcgccatttgcagtccgtgctgtcgcgcaacgagttccggaagctgttcatcgtcggcggattgctggtgggcgttggcgtctttgtggccgtcgtggtgctcaccatgctgggcgttgtggccccgtggagtggacgcttctactcgctgtgggatactggctacgccaagatccacattcccatcattgcatccgtgtcggagcatcagcccaccacttggttctcgttcttctttgatctgcacatcctggtgtgcgccttcccagtgggagtgtggtactgcatcaagcagatcaacgacgagcgcgttttcgtggtgctgtacgccatcagtgcggtttacttcgctggtgtgatggtgcgtttgatgttgaccctcacgccggtggtgtgcatgctggccggagtggccttttcgggactgttggatgtgttcctgcaagaggattcgtctaagcgaatgggcacagccataagcgcagccaccgaagtggatgaagctgaggattccattgagaagaagacgctgtacgacaaggctggcaagctgaagcatcgtactaagcatgatgcccagcaggatactggcgtcagctccaacctgaagagtattgttattttggccgttctaatgctgttgatgatgttcgctgtccactgcacgtgggtgaccagcaatgcctactccagtccctccattgtcttggctttccacaacagtcaagatggatcccgcaacattttagacgatttcagagaggcttactactggctttcgcagaacactgccgatgatgctcgcgttatgtcttggtgggattacggataccagatagcgggaatggcaaacagaacgacgctagtggataataatacgtggaacaatagtcacatagcgctggttggcaaggcaatgtcttcaaccgaggagaagtcctacgaaattatgacatctcttgacgtggactacgttttggtgatctttggcggtgtgatcggctattctggcgatgatatcaacaagttcctgtggatggtccgaattgctgagggagagcatcccaaggacattaaggaaagcgattactttaccgaccgcggtgaattcagggtagatgccgaaggtgctccggccctgctcaactgccttatgtacaaattaagctactacagattcggggaattgaagttggactacagaggtccatctggatatgatcgcacacgtaacgccgtcattgggaataaggacttcgatctgacctacctggaggaggcctacaccacagaacactggcttgttcgcatctatagggtgaagaagccgcatgagttcaatagaccatcactgaagaccaaggagagaacgattcctccagcaaacttcatttcgagaaagaactctaagcgtcgcaagggctacatacgaaaccgaccggttgttgttaagggaaaacgaaccttgaaataaacccaaaaaaaaaaaaaaaaaa

SEQ ID NO: 8 - protein, 774 aa, Drosophila melanogaster
  ref <308>: DATABASE ACCESSION NUMBER: AF132552
  ref <309>: DATABASE ENTRY DATE: 1999-04-27
  ref <313>: RELEVANT RESIDUES: (1)..(774)
MNRTPKMLNSKVAGYSSLITFAILLIAWLAGFSSRLFAVIRFESIIHEFDPWFNYRATAYMVQNGWYNFLNWFDERAWYPLGRIVGGTVYPGLMITSGGIHWLLHVLNIPVHIRDICVFLAPIFSGLTSISTYLLTKELWSAGAGLFAASFIAIVPGYISRSVAGSYDNEGIAIFALQFTYFLWVRSVKTGSVFWSAAAALSYFYMVSAWGGYVFIINLIPLHVFVLLIMGRYSPRLLTSYSTFYILGLLFSMQIPFVGFQPIRTSEHMAALGVFVLLMAVATLRHLQSVLSRNEFRKLFIVGGLLVGVGVFVAVVVLTMLGVVAPWSGRFYSLWDTGYAKIHIPIIASVSEHQPTTWFSFFFDLHILVCAFPVGVWYCIKQINDERVFVVLYAISAVYFAGVMVRLMLTLTPVVCMLAGVAFSGLLDVFLQEDSSKRMGTAISAATEVDEAEDSIEKKTLYDKAGKLKHRTKHDAQQDTGVSSNLKSIVILAVLMLLMMFAVHCTWVTSNAYSSPSIVLAFHNSQDGSRNILDDFREAYYWLSQNTADDARVMSWWDYGYQIAGMANRTTLVDNNTWNNSHIALVGKAMSSTEEKSYEIMTSLDVDYVLVIFGGVIGYSGDDINKFLWMVRIAEGEHPKDIKESDYFTDRGEFRVDAEGAPALLNCLMYKLSYYRFGELKLDYRGPSGYDRTRNAVIGNKDFDLTYLEEAYTTEHWLVRIYRVKKPHEFNRPSLKTKERTIPPANFISRKNSKRRKGYIRNRPVVVKGKRTLK

SEQ ID NO: 9 - DNA, 3094 bp, Mus musculus
  ref <308>: DATABASE ACCESSION NUMBER: NM_008408
  ref <309>: DATABASE ENTRY DATE: 2000-11-01
  ref <313>: RELEVANT RESIDUES: (1)..(3094)
ctgtcagggttgagtgcgccgctgaacggatggcagggggagcagagtgggttcctgaggagcatccgtgaggtatttgaatatcatcagttgccacccattgatgtcaagatgactaagcttggatttttgcgattgtcctatgagaagcaggacacacttctaaagcttctcatcctgtcgatggctgctgtgttatctttttctactcgtctttttgctgtgctgagatttgaaagtgtcatccatgagtttgatccgtactttaattatcggactacccggtttctggctgaggaggggttttataaattccataactggtttgatgaccgggcttggtaccctttgggccgaatcattggaggaacaatttacccaggtttaatgatcacttctgctgcaatctaccatgtactccatttcttccatatcactattgacattcggaatgtctgtgttttcctggccccacttttctcctctttcaccaccatcgttacgtaccaccttaccaaagagctcaaggatgcaggagctgggcttcttgctgctgccatgattgctgtagttcctgggtatatttctcgatctgtagctggctcctatgataatgaaggaattgctatcttttgcatgctgcttacttactacatgtggatcaaggcagtgaagactggttccatctattgggctgccaagtgtgccctcgcttatttctacatggtctcttcatggggaggctatgtgttcctgatcaacttgattcctctacatgtcctggtgctaatgctgacaggccgtttttctcaccggatctacgtagcctactgtactgtttactgcctgggcaccattctttctatgcagatttcctttgttggtttccagcccgtcctttcatcagaacacatggcagcctttggagtgtttggtctctgtcagatccatgctttcgtagattacctgcgcagcaagttgaatccacagcaattcgaagttcttttccggagtgttatctccctggttggctttgtcctcctcactgtgggagctctcctcatgctaacaggaaaaatttctccctggacagggcgtttctactctctgctggatccctcttatgctaagaataacattcccattattgcatctgtttctgagcaccagcccacaacctggtcttcctactattttgatctacagctccttgtcttcatgtttccagttggcctctattactgctttagcaacctgtctgatgctcggatttttatcatcatgtatggtgtgaccagcatgtacttttcagctgtaatggtgcgtctaatgctggtattggcacctgttatgtgcattctttctggcattggtgtttcccaggtgctgtccacatatatgaaaaatctggacataagtcgcccagacaagaagagcaagaagcaacaggattctacttaccctattaagaatgaggtggcgagtgggatgatactggtcatggctttttttctcatcacctacacgtttcattcgacttgggtgaccagtgaagcctattcttctccctccattgtactgtctgctcgtggtggggatggcagtaggatcatttttgatgacttccgagaagcgtattattggctccgtcacaatactccagaggatgcaaaagtcatgtcatggtgggattatggctaccaaattactgcaatggcaaatcggacaattttagtggacaataacacatggaataatacccatatttctcgagtagggcaggcaatggcatccacagaagaaaaagcctatgaaatcatgagggagcttgatgtcagctatgtgcttgtcatttttggaggccttactgggtattcttcggatgatatcaacaagtttctttggatggtccggattggaggaagcacagagacaggaagacacattaaggagaatgactactatactcctactggggaattccgtgttgatcgtgagggttctccggtgctgctcaactgccttatgtacaaaatgtgttactaccgctttgggcaggtctacacagaagccaagcgtccaccaggctttgaccgtgttcgaaatgctgagattggtaataaagactttgagcttgatgtcctggaggaagcgtataccacagaacactggctagtcaggatatacaaggtaaaggacctggataatcgaggcttgtcaaggacataaacgtcacattgtgccctgagcattatgcttcgcactgagcgcgtcatgttgaggacgctgaagatgttttttatatgcagtttataagaacagccggatggggttagaattgtctgcaagttttgccctggacaatatgggctgggccaagtgaaatgatttttataattctgagcaggttaccaaatgaaatgttatggctttactttggtcaattaaaagagggggggggattttttttaaatgtgccttatttgttttgacttaaattggctgatacgaggatcacagaagtgagcggatggaagaccatatccatgctctaggtccccaaatgaaccagataggagcatttttttctcctatcagcaatctcaaggactagctctggttcaacaaatgtaaacaacaactttgtcacacttttttgttttttagcacccaggtacaatgctttccttataatgggtgcttaataaatttttatcaaatgaataaatgtttctgggaccagaggagtgctgtttctgggcaagaaagacagctttcttgctgttatgtctatgttctcgatgtctatttctttagaagctctttggctttataaggacagaaagttgctgagtattcctgatctcaccagtatcctttcaaactaatggcagttattctttttctaagtagaaatgtgaagcaaaagtgactaatccagtagttcttaagatcagtgaaacatcaatcctagaggaagacactcctccaacatcaggttgatgatcagtagatgtttctggaatcagatgtcattatgtggacctacatgaagtttaggcattcaatacttcactaaacctaaaacatagta

SEQ ID NO: 10 - protein, 705 aa, Mus musculus
  ref <308>: DATABASE ACCESSION NUMBER: NP_032434
  ref <309>: DATABASE ENTRY DATE: 2000-11-01
  ref <313>: RELEVANT RESIDUES: (1)..(705)
MTKLGFLRLSYEKQDTLLKLLILSMAAVLSFSTRLFAVLRFESVIHEFDPYFNYRTTRFLAEEGFYKFHNWFDDRAWYPLGRIIGGTIYPGLMITSAAIYHVLHFFHITIDIRNVCVFLAPLFSSFTTIVTYHLTKELKDAGAGLLAAAMIAVVPGYISRSVAGSYDNEGIAIFCMLLTYYMWIKAVKTGSIYWAAKCALAYFYMVSSWGGYVFLINLIPLHVLVLMLTGRFSHRIYVAYCTVYCLGTILSMQISFVGFQPVLSSEHMAAFGVFGLCQIHAFVDYLRSKLNPQQFEVLFRSVISLVGFVLLTVGALLMLTGKISPWTGRFYSLLDPSYAKNNIPIIASVSEHQPTTWSSYYFDLQLLVFMFPVGLYYCFSNLSDARIFIIMYGVTSMYFSAVMVRLMLVLAPVMCILSGIGVSQVLSTYMKNLDISRPDKKSKKQQDSTYPIKNEVASGMILVMAFFLITYTFHSTWVTSEAYSSPSIVLSARGGDGSRIIFDDFREAYYWLRHNTPEDAKVMSWWDYGYQITAMANRTILVDNNTWNNTHISRVGQAMASTEEKAYEIMRELDVSYVLVIFGGLTGYSSDDINKFLWMVRIGGSTETGRHIKENDYYTPTGEFRVDREGSPVLLNCLMYKMCYYRFGQVYTEAKRPPGFDRVRNAEIGNKDFELDVLEEAYTTEHWLVRIYKVKDLDNRGLSRT

SEQ ID NO: 11 - DNA, 2472 bp, Homo sapiens
  ref <308>: DATABASE ACCESSION NUMBER: NM_002219
  ref <309>: DATABASE ENTRY DATE: 2000-10-31
  ref <313>: RELEVANT RESIDUES: (1)..(2472)
ctgccagggttgggtgcgccgctgaacggatggctgagggagccccgcggatcgttaggaaagccggccagctgatcgtcgtgtgttgccacccattcatgtcaagatgactaagtttggatttttgcgattgtcctatgagaagcaggacacacttttgaagcttctcattctgtcaatggctgctgtattatccttctccactcgtctgtttgctgtcctgagatttgaaagtgttatccatgagtttgatccgtactttaattatcggactaccaggttcctggctgaggaggggttttataaattccataactggtttgatgaccgagcctggtaccctttgggacgaatcattggaggaacaatttacccaggtttaatgatcacctctgctgcaatctaccatgtactccattttttccacatcaccatcgacattcggaatgtctgtgtgttcctggcccctctcttctcctccttcacctccatcgtcacgtacctccttaccaaagagctcaaggatgcaggggctgggcttcttgctgctgccatgattgctgtagttcctggatatatctcccgatctgtggctggctcctatgataatgaagggattgccatcttttgcatgctactcacctactacatgtggatcaaggcagtaaagactggttccatctgttgggcagctaagtgtgcccttgcttatttctacatggtctcgtcatggggaggttatgtgttcctgatcaacttaattcctctccacgtcctcgtgctgatgctcacaggccgtttctctcaccggatctatgtggcctactgtactgtttactgcctgggtactatactttctaggcagatctcctttgtgggtttccagcctgtcctttcatcagagcacatggcagggtttggggtctttggtctctgccagatccatgcctttgtggattacctgcgcagcaagttgaatccacaacaatttgaagttcttttccggagcgtcatctctctggtaggctttgtccttctcaccgtgggagctctcctcatgctgacaggaaaaatatctccctggacggggcgtttctactcactgctggatccctcttatgctaagaacaacatccccatcattgcttctgtgtctgagcatcagcccacaacctggtcctcatactattttgacctgcagctcctcgtcttcatgtttccagttggcctctattactgctttagcaacctgtctgatgcccggatttttatcatcatgtatggtgtgaccagcatgtacttttcagctgtaatggtgcgtctaatgctagtgttggcacctgttatgagcattctctctggcattggagtctcccaggtgctgtccacatacatgaagaatctggacataagtcgcccagacaagaagagcaagaagcaacaggattccacctaccctattaagattgaagtggcaagtgggatgatactggtcatggctttctttctcatcacctacacctttcattcaacctgggtgaccagtgaggcctactcttctccgtccattgtactatctgcccgtggtggggatggcagtaggatcatatttgatgacttccgagaagcatattattggcttcgtcataatactccagaggatgcgaaggtcatgtcctggtgggattatggctatcagattacagctatggcaaaccgaacaattttagtggacaataacacatggaataatacccatatttctcgagtagggcaggcaatggcgtccacagaggaaaaagcctatgagatcatgagggagctcgatgtcagctatgtgctggtcatttttggaggcctcactgggtattcctctgatgatatcaacaagtttctttggatggtccggattggagggagcacagatacaggcaaacatatcaaggagaatgactattatactccaactggggagttccgtgtggaccgtgaaggttctccagtgctgctcaactgcctcatgtacaagatgtgttactatcgctttggacaggtttacacagaagccaagcgtcctccaggctttgaccgtgtccgaaatgctgagattgggaataaagactttgagcttgatgtcctggaggaaggctataccacagaacattggctggtcaggatatacaaggtaaaggacctggataatcgaggcttgtcaaggacataaatgtcacgtccagctctgatatcttcgcactgagcacatcacatttaggacgttgaagattttttttttttttttttttttaatatgcagtttgtaagaacaaaactggatggcatccgaattgtctggaagttttgtcttgggcatgatgggctgggccaaatgaaatgatttttataattctaaacaggttaccaaatgaaatgtcatggctttactttggtcaattaaaggggggaattttttta

SEQ ID NO: 12 - protein, 705 aa, Homo sapiens
  ref <308>: DATABASE ACCESSION NUMBER: NP_002210
  ref <309>: DATABASE ENTRY DATE: 2000-10-31
  ref <313>: RELEVANT RESIDUES: (1)..(705)
MTKFGFLRLSYEKQDTLLKLLILSMAAVLSFSTRLFAVLRFESVIHEFDPYFNYRTTRFLAEEGFYKFHNWFDDRAWYPLGRIIGGTIYPGLMITSAAIYHVLHFFHITIDIRNVCVFLAPLFSSFTSIVTYLLTKELKDAGAGLLAAAMIAVVPGYISRSVAGSYDNEGIAIFCMLLTYYMWIKAVKTGSICWAAKCALAYFYMVSSWGGYVFLINLIPLHVLVLMLTGRFSHRIYVAYCTVYCLGTILSRQISFVGFQPVLSSEHMAGFGVFGLCQIHAFVDYLRSKLNPQQFEVLFRSVISLVGFVLLTVGALLMLTGKISPWTGRFYSLLDPSYAKNNIPIIASVSEHQPTTWSSYYFDLQLLVFMFPVGLYYCFSNLSDARIFIIMYGVTSMYFSAVMVRLMLVLAPVMSILSGIGVSQVLSTYMKNLDISRPDKKSKKQQDSTYPIKIEVASGMILVMAFFLITYTFHSTWVTSEAYSSPSIVLSARGGDGSRIIFDDFREAYYWLRHNTPEDAKVMSWWDYGYQITAMANRTILVDNNTWNNTHISRVGQAMASTEEKAYEIMRELDVSYVLVIFGGLTGYSSDDINKFLWMVRIGGSTDTGKHIKENDYYTPTGEFRVDREGSPVLLNCLMYKMCYYRFGQVYTEAKRPPGFDRVRNAEIGNKDFELDVLEEGYTTEHWLVRIYKVKDLDNRGLSRT

SEQ ID NO: 13 - protein, 757 aa, Caenorhabditis elegans
  ref <308>: DATABASE ACCESSION NUMBER: P46975
  ref <309>: DATABASE ENTRY DATE: 1996-10-01
  ref <313>: RELEVANT RESIDUES: (1)..(757)
MTSTTAARTASSRVGATTLLTIVVLALAWFVGFASRLFAIVRFESIIHEFDPWFNYRATHHMVQHGFYKFLNWFDERAWYPLGRIVGGTVYPGLMVTSGLIHWILDSLNFHVHIREVCVFLAPTFSGLTAIATYLLTKELWSPGAGLFAACFIAISPGYTSRSVAGSYDNEGIAIFALQFTYYLWVKSLKTGSIMWASLCALSYFYMVSAWGGYVFIINLIPLHALALIIMGRYSSRLFVSYTSFYCLATILSMQVPFVGFQPVRTSEHMPAFGVFGLLQIVALMHYARNRITRQQFMTLFVGGLTILGALSVVVYFALVWGGYVAPFSGRFYSLWDTGYAKIHIPIIASVSEHQPTTWVSFFFDLHITAAVFPVGLWYCIKKVNDERVFIILYAVSAVYFAGVMVRLMLTLTPAVCVLAGIGFSYTFEKYLKDEETKERSSSQSGTTKDEKLYDKAAKNVKSRNANDGDESGVSSNVRTIISIILVIFLLMFVVHATYVTSNAYSHPSVVLQSSTNNGDRIIMDDFREAYHWLRENTADDARVMSWWDYGYQIAGMANRTTLVDNNTWNNSHIALVGKAMSSNESAAYEIMTELDVDYILVIFGGVIGYSGDDINKFLWMVRIAQGEHPKDIREENYFTSTGEYSTGAGASETMLNCLMYKMSYYRFGETRVGYNQAGGFDRTRGYVIGKKDITLEYIEEAYTTENWLVRIYKRKKLPNRPTVKSEEATIPIKGKKATQGKNKKGVIRPAPTASKA

(19) United States
(12) Patent Application Publication
(10) Pub. No.: US 2003/0148285 A1
(43) Pub. Date: Aug. 7, 2003

(54) Mammalian SIMP protein, gene sequence and uses thereof in cancer therapy

(76) Inventors: Claude Perreault, Outremont (CA); Kevin McBride, Montreal (CA)

Correspondence Address: MYERS BIGEL SIBLEY & SAJOVEC, PO BOX 37428, RALEIGH, NC, 27627, US

(21) Appl. No.: 10/028,384

(22) Filed: Dec. 20, 2001

(51) Int. Cl. C12Q 1/68; G01N 33/574; A01K 67/00; C07H 21/04; C12P 21/02; C12N 5/06; C07K 16/30; A61K 48/00

(52) U.S. Cl. 435/6; 424/155.1; 514/44; 435/7.23; 435/69.1; 435/226; 435/325; 435/320.1; 800/8; 536/23.2; 530/388.8

(57) ABSTRACT

This invention provides SIMP nucleic acid and sequences. Also provided are methods for using SIMP nucleic acids, proteins, fragments, antibodies, probes, and cells, to characterize SIMP, modulate SIMP cellular levels, modulate immune responses and diagnose and treat cancers.

### BACKGROUND OF THE INVENTION

a) Field of the Invention

The present invention is concerned with a protein called “SIMP” that is a Source of Immunodominant MHC-associated Peptides and more particularly to the use of SIMP nucleic acids, proteins, fragments, antibodies, probes, and cells, to characterize SIMP, modulate its cellular levels, diagnose and treat cancers and modulate an immune response.

b) Brief Description of the Prior Art

Adoptive immunotherapy is a main approach that is currently being investigated in the field of cancer immunotherapy. Adoptive immunotherapy involves injection of lymphocytes (or of lymphocyte receptor(s) transfected into another cell type) from one individual to an other. According to this approach, patients with cancer are treated by allogeneic hematopoietic cell transplant (AHCT) from a cancer-free donor. Following AHCT, eradication of cancer cells is primarily mediated by a donor T-cell dependent immune reaction commonly referred to as the graft-versus-tumor (GVT) effect.

Recently, one of the present inventors has shown that it is possible to transfer T-cells from a donor to a compatible recipient without causing to the latter a graft-versus-host disease (GVHD) reaction (International PCT application PCT/CA01/01477; and Fontaine et al., (2001). Nat. Med. 7:789-794). These experiments, which were carried out in mice, were based on the priming of T-cells specifically reacting against B6^{dom1}, a selected immunodominant ubiquitous MiHA. Although the immunogenic properties of B₆^{dom1} have been characterised (Eden et al., (1999) J. Immunol. 162:4502-4510), the identity of the gene/protein from which B6^{dom1} was derived and whether a human homolog existed was unknown until now.

Given that B6^{dom1} peptide(s) seemed to represent an ideal target for adoptive cancer immunotherapy, there is thus a need to identify the human homolog of B6^{dom1}.

There is also a need for a human protein and a nucleic acid encoding the same, that is expressed ubiquitously in human cells and which has the potential of generating a plurality of protein fragments binding with high affinity to human MHC molecules, and more particularly human HLA molecules.

The present invention fulfils this need and also other needs as it will be apparent to those skilled in the art upon reading the following specification.

### SUMMARY OF THE INVENTION

The present inventors have discovered a protein called “SIMP” (Source of Immunodominant MHC-associated Peptides) which is a human homolog of the mouse gene encoding B6^{dom1}. The present inventors have also discovered uses for human SIMP proteins, fragments, nucleic acids, and antibodies for modulating its cellular levels, for diagnosing and treating cancers, and for modulating immune response

In general, the invention features an isolated or purified nucleic acid molecule, such as genomic, cDNA, antisense DNA, RNA or a synthetic nucleic acid molecule that encodes or corresponds to a human SIMP polypeptide.

According to a first aspect, the invention features isolated or purified nucleic acid molecules, polynucleotides, polypeptides, human proteins and fragment thereof.

In a first embodiment, the isolated or purified nucleic acid molecule encodes a human protein that is expressed ubiquitously in human cells, the protein having the potential of generating a plurality of protein fragments binding with high affinity to a human HLA molecule. Preferably, the HLA molecule is selected from the HLA molecules listed in Table 1. Preferably, the protein fragments are selected from the peptides listed in Table 1 as well.

In another embodiment, the invention provides an isolated or purified human protein that is expressed ubiquitously in human cells, the protein having the potential of generating a plurality of protein fragments that bind with high affinity to a human HLA molecule. In further embodiments, there is provided polypeptides comprising a definite amino acid sequence.

In preferred embodiments of the invention, the human protein is overexpressed in proliferative cells, such as tumoral cells, and expression of the protein is essential for the tumoral cell's survival. More preferably, the human protein is a functional or structural homolog of yeast STT3 (SEQ ID NO: 6) and/or a paralog of human ITM1 (SEQ ID NO: 12).

According to a specific embodiment, the nucleic acid of the invention comprises a polynucleotide having a nucleotide sequence coding an amino acid sequence selected from the group consisting of:

a) an amino acid sequence having greater than 71% amino acid sequence identity to SEQ ID NO:8;

b) an amino acid sequence having greater than 71% amino acid sequence identity to an amino acid sequence encoded by an open reading frame having SEQ ID NO:7;

c) an amino acid sequence having greater than 82% amino acid sequence homology to SEQ ID NO: 8;

d) an amino acid sequence having greater than 82% amino acid sequence homology to an amino acid sequence encoded by an open reading frame having SEQ ID NO: 7;

e) an amino acid sequence having greater than 97% amino acid sequence identity to SEQ ID NO: 2;

f) an amino acid sequence having greater than 97% amino acid sequence identity to an amino acid sequence encoded by an open reading frame having SEQ ID NO: 1;

g) an amino acid sequence having greater than 97% amino acid sequence homology to SEQ ID NO: 2; and

h) an amino acid sequence having greater than 97% amino acid sequence homology to an amino acid sequence encoded by an open reading frame having SEQ ID NO: 1.

More preferably, the nucleic acid comprises a polynucleotide having a nucleotide sequence coding an amino acid sequence 100% identical to SEQ ID NO: 2 and/or 100% identical to an amino acid sequence encoded by an open reading frame having SEQ ID NO: 1.

According to another specific embodiment, the nucleic acid of the invention comprises a polynucleotide having a nucleotide sequence selected from the group consisting of:

a) a nucleotide sequence having greater than 63% nucleotide sequence identity with SEQ ID NO:7;

b) a nucleotide sequence having greater than 63% nucleotide sequence identity with a nucleic acid encoding an amino acid sequence of SEQ ID NO:8;

c) a nucleotide sequence having at least 91% nucleotide sequence identity with SEQ ID NO: 1; and

d) a nucleotide sequence having at least 91% nucleotide sequence identity with a nucleic acid encoding an amino acid sequence of SEQ ID NO: 2.

More preferably, the nucleic acid comprises a polynucleotide 100% identical to SEQ ID NO: 1.

According to another aspect, the invention features an isolated or purified nucleic acid molecule which comprises a polynucleotide having a definite nucleotide sequence selected from the group consisting of:

a) a nucleotide sequence having greater than 63% nucleotide sequence identity with SEQ ID NO: 7;

b) a nucleotide sequence having greater than 63% nucleotide sequence identity with a nucleic acid encoding an amino acid sequence of SEQ ID NO:8;

c) a nucleotide sequence having at least 91% nucleotide sequence identity with SEQ ID NO: 1;

d) a nucleotide sequence having at least 91% nucleotide sequence identity with a nucleic acid encoding an amino acid sequence of SEQ ID NO: 2; and

e) a nucleotide sequence complementary to any of the nucleotide sequences in (a), (b), (c) or (d).

Preferably the nucleic acid molecule comprises a polynucleotide having a nucleotide sequence selected from the group consisting of:

a) a nucleotide sequence having at least 91% nucleotide sequence identity with SEQ ID NO: 1;

b) a nucleotide sequence having at least 91% nucleotide sequence identity with a nucleic acid encoding an amino acid sequence of SEQ ID NO: 2; and

c) a nucleotide sequence complementary to any of the nucleotide sequences in (a) or (b).

More preferably, the nucleic acid molecule comprises a polynucleotide having:

a) a nucleotide sequence 100% identical to SEQ ID NO: 1;

b) a nucleotide sequence complementary to SEQ ID NO: 1; and/or

c) at least 15 nucleotides of the polynucleotide of (a) or (b).

In a related aspect, the invention features an isolated or purified nucleic acid molecule which hybridizes under low, preferably high, stringency conditions to any of the nucleic acid molecules mentioned hereinabove.

In a more specific aspect, the invention features an isolated or purified human nucleic acid molecule comprising a polynucleotide having the SEQ ID NO: 1, or degenerate variants thereof, and encoding a human SIMP polypeptide. Preferably, the nucleic acid is a cDNA and it encodes the amino acid sequence of SEQ ID NO: 2 or a fragment thereof.

The invention also features substantially pure human polypeptides and proteins that are encoded by any of the above mentioned nucleic acids. In a preferred embodiment, the invention aims at an isolated or purified polypeptide comprising an amino acid sequence selected from the group consisting of:

a) an amino acid sequence having greater than 71% amino acid sequence identity to SEQ ID NO: 8;

b) an amino acid sequence having greater than 71% amino acid sequence identity to an amino acid sequence encoded by an open reading frame having SEQ ID NO: 7;

c) an amino acid sequence having greater than 82% amino acid sequence homology to SEQ ID NO: 8;

d) an amino acid sequence having greater than 82% amino acid sequence homology to an amino acid sequence encoded by an open reading frame having SEQ ID NO: 7;

e) an amino acid sequence having greater than 97% amino acid sequence identity to SEQ ID NO: 2;

f) an amino acid sequence having greater than 97% amino acid sequence identity to an amino acid sequence encoded by an open reading frame having SEQ ID NO: 1;

g) an amino acid sequence having greater than 97% amino acid sequence homology to SEQ ID NO: 2; and

h) an amino acid sequence having greater than 97% amino acid sequence homology to an amino acid sequence encoded by an open reading frame having SEQ ID NO: 1

More preferably, the polypeptide comprises an amino acid sequence selected from the group consisting of:

a) an amino acid sequence 100% identical to SEQ ID NO: 2;

b) an amino acid sequence 100% identical to an amino acid sequence encoded by an open reading frame having SEQ ID NO: 1; and

c) an amino acid sequence consisting of at least eight consecutive amino acids of (a) or (b).

In an even more specific aspect, the invention features a substantially pure human SIMP polypeptide, or a fragment thereof. Preferably, the SIMP polypeptide or fragment comprises an amino acid sequence having greater than 97% amino acid sequence homology, and more preferably 100%, with a polypeptide selected from the group consisting of:

a) a polypeptide having SEQ ID NO: 2;

b) a polypeptide having an amino acid sequence encoded by an open reading frame having SEQ ID NO: 1; and

c) a polypeptide that is a fragment of (a) or (b).

In a related aspect, the invention features an isolated or purified human protein that is a paralog of a human protein having SEQ ID NO:12. Preferably the protein comprises an amino acid sequence having at least 25% identity or at least 25% homology with SEQ ID NO:12. Even more preferably, the percentages of identity and homology are of at least 50% and more specifically of about 56% and 59% respectively.

The present invention also features protein fragments derived from any of the above mentioned protein or polypeptides. Accordingly, the present invention encompasses each of the polypeptides fragment listed in Table 1 and any fragment comprising at least eight sequential amino acids of SEQ ID NO:2 (hSIMP) or of SEQ ID NO:12 (hITM1). Similarly, the invention further encompasses polypeptides fragment of comprising an amino acid sequence encoded by a nucleotide sequence comprising at least 24 sequential nucleic acid of SEQ ID NO:1 (hSIMP) or of SEQ ID NO:11 (hITM1).

The present invention further features an antisense nucleic acid and a pharmaceutical composition comprising the same. According to a first embodiment, the antisense hybridizes under high stringency condition to SEQ ID NO: 1 or to a complementary sequence thereof. According to another embodiment, the antisense hybridizes under high stringency conditions to a genomic sequence or to a mRNA so that it reduces human SIMP cellular levels of expression. Preferably, the antisense is complementary to a nucleic acid sequence encoding a protein having SEQ ID NO:1 or encoding a fragment of this protein.

In a related aspect, the present invention further features a method for modulating tumoral cell survival or for eliminating a tumoral cell in a mammal, the method comprising the step of reducing cellular expression levels of a SIMP polypeptide. Preferably, the method comprises the step of delivering a human SIMP antisense into the tumoral cell.

Furthermore, the present invention features a method for eliminating tumoral cells in a mammal, preferably a human. The method comprises the step of injecting, into the mammal's circulatory system, T-lymphocytes that recognize a immune complex that is present at the surface of the tumoral cells, the immune complex consisting of a SIMP protein fragment or a ITM1 protein fragment bound to an MHC molecule. Preferably, the immune complex consists of a human SIMP protein fragment bound to a HLA molecule, the human SIMP protein fragment comprising at least eight sequential amino acids of SEQ ID NO: 2. Even more preferably, the hSIMP protein fragment is selected from the peptides listed in Table 1.

The present invention also features a method for increasing cell proliferation in a mammal, comprising the step of: i) contacting the cell with a SIMP polypeptide; and/or ii) increasing cellular expression levels of a SIMP polypeptide.

The present invention further features a method for modulating an immune response in a mammal, preferably a human, comprising increasing the cellular expression levels of a SIMP polypeptide in the lymphoid cells of the mammals. In a preferred embodiment, the method is used for increasing the level and/or the duration of an antigen-primed lymphocyte proliferation. Preferably, the method comprises the transfection of lymphocytes with a cDNA coding for a SIMP polypeptide.

The present invention features also a method for decreasing lymphoid cells proliferation, comprising decreasing in these cells cellular expression levels of a SIMP polypeptide. In a preferred embodiment, the method is used for suppressing an immune response responsible for an autoimmune disease or a transplant rejection. Preferably, the method comprises the delivery of a SIMP antisense into the lymphoid cells.

According to another aspect, the invention features a nucleotide probe comprising a sequence of at least 15 sequential nucleotides of SEQ ID NO: 1 or of a sequence complementary to SEQ ID NO:1. The invention also encompasses a substantially pure nucleic acid that hybridizes under low, preferably high, stringency conditions to a probe of at least 40 nucleotides in length that is derived from SEQ ID NO:1.

According to another aspect, the invention features a purified antibody. In a preferred embodiment, the antibody specifically binds to a purified mammalian SIMP polypeptide. Preferably, the antibody binds to a polypeptide having an amino acid sequence selected from the group consisting of SEQ ID NO: 2 and SEQ ID NO: 4. In another embodiment, the invention provides a monoclonal or polyclonal antibody which recognizes any of the human SIMP proteins, polypeptides, or fragments defined hereinabove.

According to a further aspect, the invention features a method for determining the amount of a SIMP polypeptide in a biological sample, the method comprising the step of contacting the sample with an antibody or with a probe as defined previously.

In a related aspect, the invention features a method of diagnosis of a cancer in a human subject. The method comprises the step of determining the amount of a human SIMP polypeptide in a cell or a biological sample from a human subject, wherein the amount of SIMP is indicative of a probability for this subject to harbor proliferating tumoral cells. The method is particularly useful for detecting proliferating tumoral cells that grow rapidly and display a short doubling time. Such tumoral cells are commonly found in lung cancers, intestine cancers, sarcomas, prostate cancer, testis cancer, breast cancer, melanomas, pancreatic cancer prostate cancer and hematologic cancers.

In another related aspect, the invention features a kit for determining the amount of a SIMP polypeptide in a sample, the kit comprising an antibody or a probe as defined previously, and at least one element selected from the group consisting of instructions for using the kit, reaction buffer(s), and enzyme(s).

The nucleic acids of the invention may be incorporated into a vector and or a cell (such as a mammalian, yeast, nematode or bacterial cell). The nucleic acids may also be incorporated into a transgenic animal or embryo thereof. Therefore, the present invention features cloning or expression vectors, transformed or transfected cells and transgenic animals that contain any of the nucleic acids of the invention and more particularly those encoding a SIMP protein, polypeptide or fragment.

In a related aspect, the invention features a method for producing a human SIMP polypeptide comprising:

providing a cell transformed with a nucleic acid sequence encoding a human SIMP polypeptide positioned for expression in this cell;

culturing the transformed cell under conditions suitable for expressing the nucleic acid; and

producing the hSIMP polypeptide.

One of the greatest advantages of the present invention is that it provides nucleic acid molecules, proteins, polypeptides, antibodies, probes, and cells that can be used for characterizing SIMP, modulate its cellular levels, diagnose and treat cancers and modulate an immune response.

Other objects and advantages of the present invention will be apparent upon reading the following non-restrictive description of the preferred embodiments thereof and from the claims.

### BRIEF DESCRIPTION OF THE DRAWINGS

FIG. 1 is a graph showing the assessment of peptide recognition by C3H.SW anti-C57BL/6 cytotoxic T-lymphocytes (CTLs).

### DETAILED DESCRIPTION OF THE INVENTION

A) Definitions

Throughout the text, the word “kilobase” is generally abbreviated as “kb”, the words “deoxyribonucleic acid” as “DNA”, the words “ribonucleic acid” as “RNA”, the words “complementary DNA” as “cDNA”, the words “polymerase chain reaction” as “PCR”, and the words “reverse transcription” as “RT”. Nucleotide sequences are written in the 5′ to 3′ orientation unless stated otherwise.

In order to provide an even clearer and more consistent understanding of the specification and the claims, including the scope given herein to such terms, the following definitions are provided:

Antisense: as used herein in reference to nucleic acids, is meant a nucleic acid sequence, regardless of length, that is complementary to the coding strand of a gene.

Expression: refers to the process by which gene encoded information is converted into the structures present and operating in the cell. In the case of cDNAs, cDNA fragments and genomic DNA fragments, the transcribed nucleic acid is subsequently translated into a peptide or a protein in order to carry out its function if any. The terms “overexpression” refer to an upward deviation respectively in assayed levels of expression as compared to a baseline expression level which is the level of expression that is found under normal conditions and normal level of functioning (e.g. non tumoral cells). By “positioned for expression” is meant that the DNA molecule is positioned adjacent to a DNA sequence which directs transcription and translation of the sequence (i.e., facilitates the production of, e.g., a NAIP polypeptide, a recombinant protein or a RNA molecule).

Fragment: Refers to a section of a molecule, such as a protein, a polypeptide or a nucleic acid, and is meant to refer to any portion of the amino acid or nucleotide sequence.

Homolog: refers to a nucleic acid molecule or polypeptide that shares similarities in DNA or protein sequences.

Host: A cell, tissue, organ or organism capable of providing cellular components for allowing the expression of an exogenous nucleic acid embedded into a vector or a viral genome, and for allowing the production of viral particles encoded by such vector or viral genome. This term is intended to also include hosts which have been modified in order to accomplish these functions. Bacteria, fungi, animal (cells, tissues, or organisms) and plant (cells, tissues, or organisms) are examples of a host.

Isolated or Purified or Substantially pure: Means altered “by the hand of man” from its natural state, i.e., if it occurs in nature, it has been changed or removed from its original environment, or both. For example, a polynucleotide or a protein/peptide naturally present in a living organism is not “isolated”, the same polynucleotide separated from the coexisting materials of its natural state, obtained by cloning, amplification and/or chemical synthesis is “isolated” as the term is employed herein. Moreover, a polynucleotide or a protein/peptide that is introduced into an organism by transformation, genetic manipulation or by any other recombinant method is “isolated” even if it is still present in said organism.

Nucleic acid: Any DNA, RNA sequence or molecule having one nucleotide or more, including nucleotide sequences encoding a complete gene. The term is intended to encompass all nucleic acids whether occurring naturally or non-naturally in a particular cell, tissue or organism. This includes DNA and fragments thereof, RNA and fragments thereof, cDNAs and fragments thereof, expressed sequence tags, artificial sequences including randomized artificial sequences.

Open reading frame (“ORF”): The portion of a cDNA that is translated into a protein. Typically, an open reading frame starts with an initiator ATG codon and ends with a termination codon (TM, TAG or TGA).

Paralog: As used herein, refers to a protein or a polypeptide that is encoded by a gene locus that has arisen through evolution by gene duplication in one species.

Polypeptide: means any chain of more than two amino acids, regardless of post-translational modification such as glycosylation or phosphorylation.

SIMP nucleic acid: means any nucleic acid (see above) encoding a mammalian polypeptide that has the potential of generating a plurality of protein fragments binding with high affinity to MHC molecules, and having at least 90%, preferably at least 95% and most preferably 100% identity or homology to the amino acid sequence shown in SEQ. ID. NO: 2 (human) or 4 (mouse). When referring to a human SIMP nucleic acid, the nucleic acid encoding SEQ. ID. NO: 2 is more particularly concerned. SIMP protein or SIMP polypeptide: means a polypeptide, or fragment thereof, encoded by a SIMP nucleic acid as described above.

Specifically binds: means an antibody that recognizes and binds a protein but that does not substantially recognize and bind other molecules in a sample, e.g., a biological sample, that naturally includes protein.

Substantially identical: means a polypeptide or nucleic acid exhibiting at least 50%, preferably 85%, more preferably 90%, and most preferably 95% homology to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides. Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Owl 53705). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine. More particularly, “substantially pure polypeptide” means a polypeptide that has been separated from the components that naturally accompany it. Typically, the polypeptide is substantially pure when it is at least 60%, by weight, free from the proteins and naturally-occurring organic molecules with which it is naturally associated. Preferably, the polypeptide is a SIMP polypeptide that is at least 75%, more preferably at least 90%, and most preferably at least 99%, by weight, pure. A substantially pure SIMP polypeptide may be obtained, for example, by extraction from a natural source (e.g. a fibroblast, neuronal cell, or lymphocyte) by expression of a recombinant nucleic acid encoding a NAIP polypeptide, or by chemically synthesizing the protein. Purity can be measured by any appropriate method, e.g., by column chromatography, polyacrylamide gel electrophoresis, or HPLC analysis. A protein is substantially free of naturally associated components when it is separated from those contaminants which accompany it in its natural state. Thus, a protein which is chemically synthesized or produced in a cellular system different from the cell from which it naturally originates will be substantially free from its naturally associated components. Accordingly, substantially pure polypeptides include those derived from eukaryotic organisms but synthesized in E. coli or other prokaryotes. By “substantially pure DNA” is meant DNA that is free of the genes which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding an additional polypeptide sequence.

Transformed or Transfected or Transgenic cell: refers to a cell into which (or into an ancestor of which) has been introduced, by means of recombinant DNA techniques, a DNA molecule encoding (as used herein) a SIMP polypeptide. By “transformation” is meant any method for introducing foreign molecules into a cell. Lipofection, calcium phosphate precipitation, retroviral delivery, electroporation, and ballistic transformation are just a few of the teachings which may be used.

Transgenic animal: any animal having a cell which includes a DNA sequence which has been inserted by artifice into the cell and becomes part of the genome of the animal which develops from that cell. As used herein, the transgenic animals are usually mammalian (e.g., rodents such as rats or mice) and the DNA (transgene) is inserted by artifice into the nuclear genome.

Ubiquitously expressed: refers to a polypeptide that is present, under normal conditions, in every single cell of an organism.

Vector: A self-replicating RNA or DNA molecule which can be used to transfer an RNA or DNA segment from one organism to another. Vectors are particularly useful for manipulating genetic constructs and different vectors may have properties particularly appropriate to express protein(s) in a recipient during cloning procedures and may comprise different selectable markers. Bacterial plasmids are commonly used vectors.

B) General Overview of the Invention

The present inventors have discovered a protein called “SIMP” (Source of Immunodominant MHC-associated Peptides). In human, this protein is the homolog of the mouse gene encoding B6^{dom1} (referred herein as mouse SIMP). The human SIMP is also a paralog of human ITM1. The present inventors have also discovered uses for human SIMP proteins, fragments, nucleic acids, and antibodies for modulating its cellular levels and for diagnosing and treating cancers. Each of the aspects of the invention will be described in details hereinafter.

i) Cloning and Molecular Characterization of SIMP

As it will be described hereinafter in the exemplification section of the invention, the inventors have discovered, cloned and sequenced a human cDNA encoding a new human protein called human SIMP. This procedure was carried out starting with the amino acid sequence of a mouse minor histocompatibility antigen (MiHA) called “B6^{dom1}”.

The sequence of the SIMP cDNA and predicted amino acid sequence is shown in the “Sequence Listing” section. SEQ ID NO: 1 corresponds to the human SIMP cDNA and SEQ ID NO: 2 corresponds to the predicted amino acid sequence of the human protein.

The hSIMP gene encodes a protein of 826 amino acids long. In silico analysis indicates that human SIMP protein has the following features: it has a molecular weight of about 93 674 g/mol, an isoelectric point of about 9.0; an instability index of about 41 (i.e. unstable); an aliphatic index of about 88; and a grand average of hydropathicity (GRAVY) of about 0.038. It further comprises many potential phosphorylation sites (26 Ser, 9 Thr, and 9 Tyr); and also many potential N-glycosylation and myristoylation sites. It also possesses more than 10 potential transmembrane domains.

As shown herein below, hSIMP protein contains an amino acid sequence having the potential of generating numerous peptides or peptide fragments possessing a high binding affinity motif for HLA class I molecules. This is very interesting since some but not all proteins generate peptides that are presented by MHC molecules. The most important factor determining whether a given peptide sequence will be presented by MHC molecules is its affinity for MHC molecules expressed by the cell in which it is expressed. Thus, a peptide with a low affinity for relevant MHC molecules will not form significant amounts of MHC/peptide complexes at the cell surface. On the contrary, the probability that a peptide with a high affinity for relevant MHC molecules will form significant levels of MHC/peptide complexes is about 68%. This is largely due to the fact that MHC class I molecules serve as templates for guiding ER aminopeptidases to generate the optimal MHC class I binding epitopes. In this way, the antigen-processing pathway efficiently generates peptides that fit exactly within the antigen binding grooves of the MHC class I molecules. Peptide sequences in a given protein that have a high affinity for a specific HLA molecule can be predicted with the BIMAS™ algorithm (http://bimas.dcrt.nuh.gov/molbio/hla bind/index.html!). The validity of predictions based on this program has been confirmed in about fifty studies.

Strikingly, many hSIMP peptides sequences possess a high affinity binding motif for HLA class I molecules. Those with the highest affinity are listed in Table 1. Methods of use of these peptides are described in the following sections.

TABLE 1
Human SIMP-derived peptides with a high affinity binding motif
for HLA molecules

| HLA molecule | Mers | Position | Sequence | Score |
|---|---|---|---|---|
| A1 | 10 | 1 | MAEPSAPESK | 180.000 |
| A_0201 | 9 | 544 | LMLLMMFAV | 4214.897 |
| | | 303 | ILSMQIPFV | 1495.716 |
| | | 329 | ALLQAYAFL | 652.087 |
| | | 459 | RLMLTLTPV | 591.888 |
| | | 71 | LLSFTILFL | 459.398 |
| | | 543 | MLMLLMMFA | 395.296 |
| | | 271 | NLIPLHVFV | 382.536 |
| | | 81 | WLAGFSSRL | 373.415 |
| | | 230 | LQFTYYLWV | 365.936 |
| | | 235 | YLWVKSVKT | 284.517 |
| | | 349 | FQTLFFLGV | 234.204 |
| | | 435 | NINDERVFV | 215.655 |
| | | 291 | YIAYSTFYI | 210.500 |
| | | 428 | GLWFCIKNI | 199.162 |
| | | 172 | FLAPTFSGL | 186.707 |
| | | 460 | LMLTLTPVV | 129.543 |
| | | 546 | LLMMFAVHC | 118.745 |
| | | 509 | NLYDKAGKV | 118.628 |
| | | 156 | ILNTLNITV | 118.238 |
| | | 358 | SLAAGAVFL | 117.493 |
| | | 179 | GLTSISTFL | 117.493 |
| | | 347 | QEFQTLFFL | 112.763 |
| | | 228 | FALQFTYYL | 105.542 |
| | 10 | 543 | MLMLLMMFAV | 5836.011 |
| | | 548 | MMFAVHCTWV | 1737.776 |
| | | 70 | SLLSFTILFL | 999.867 |
| | | 302 | LILSMQIPFV | 760.945 |
| | | 229 | ALQFTYYLWV | 573.804 |
| | | 386 | SLWDTGYAKI | 532.542 |
| | | 281 | LLMQRYSKRV | 437.482 |
| | | 365 | FLSVIYLTYT | 433.632 |
| | | 199 | LLAACFIAIV | 423.695 |
| | | 542 | LMLMLLMMFA | 285.492 |
| | | 470 | MLSAIAFSNV | 224.653 |
| | | 331 | LQAYAFLQYL | 176.996 |
| | | 258 | YMVSAWGGYV | 165.213 |
| | | 155 | WILNTLNITV | 162.769 |
| | | 420 | ILVCTFPAGL | 138.001 |
| | | 179 | GLTSISTFLL | 123.902 |
| | | 545 | MLLMMFAVHC | 118.745 |
| | | 271 | NLIPLHVFVL | 116.840 |
| | | 71 | LLSFTILFLA | 112.664 |
| | | 546 | LLMMFAVHCT | 107.808 |
| | | 459 | RLMLTLTPVV | 105.510 |
| | | 409 | TTWVSFFFDL | 103.124 |
| A_0205 | 10 | 266 | YVFIINLIPL | 252.000 |
| A3 | 9 | 386 | SLWDTGYAK | 300.000 |
| A24 | 9 | 561 | AYSSPSVVL | 200.000 |
| | | 722 | YYRFGEMQL | 200.000 |
| | | 807 | GYIKNKLVF | 150.000 |
| | | 265 | GYVFIINLI | 126.000 |
| | | 694 | DYFTPQGEF | 110.000 |
| | | 445 | LYAISAVYF | 100.000 |
| | | 717 | MYKMSYYRF | 100.000 |
| | 10 | 451 | VYFAGVMVRL | 280.000 |
| | | 293 | AYSTFYIVGL | 200.000 |
| | | 721 | SYYRFGEMQL | 200.000 |
| | | 375 | GYIAPWSGRF | 150.000 |
| | | 666 | GYSGDDINKF | 132.000 |
| A68.1 | 9 | 642 | ETAAYKIMR | 300.000 |
| | 10 | 276 | HVFVLLLMQR | 400.000 |
| | | 450 | AVYFAGVMVR | 200.000 |
| | | 786 | RVTNIFPKQK | 120.000 |
| | | 733 | RTPPGFDRTR | 112.500 |
| | | 158 | NTLNITVHIR | 100.000 |
| B7 | 9 | 54 | APAGLSGGL | 240.000 |
| | 10 | 378 | APWSGRFYSL | 240.000 |
| | | 49 | APPKPAPAGL | 240.000 |
| B8 | 10 | 747 | GNKDIKFKHL | 120.000 |
| | 8 | 8 | ESKHKSSL | 160.000 |
| B14 | 9 | 284 | QRYSKRVYI | 100.000 |
| | 10 | 439 | ERVFVALYAI | 108.000 |
| | | 284 | QRYSKRVYIA | 100.000 |
| B_2702 | 9 | 284 | QRYSKRVYI | 300.000 |
| | | 599 | ARVMSWWDY | 200.000 |
| | | 87 | SRLFAVIRF | 200.000 |
| | | 135 | GRIVGGTVY | 200.000 |
| | | 805 | KRGYIKNKL | 180.000 |
| | | 382 | GRFYSLWDT | 100.000 |
| | 10 | 93 | IRFESIIHEF | 1000.000 |
| | | 723 | YRFGEMQLDF | 1000.000 |
| | | 288 | KRVYIAYSTF | 600.000 |
| | | 340 | LRDRLTKQEF | 200.000 |
| | | 284 | QRYSKRVYIA | 100.000 |
| B_2705 | 9 | 805 | KRGYIKNKL | 6000.000 |
| | | 284 | QRYSKRVYI | 3000.000 |
| | | 741 | TRNAEIGNK | 2000.000 |
| | | 584 | FREAYFWLR | 1000.000 |
| | | 87 | SRLFAVIRF | 1000.000 |
| | | 135 | GRIVGGTVY | 1000.000 |
| | | 732 | FRTPPGFDR | 1000.000 |
| | | 577 | TRNILDDFR | 1000.000 |
| | | 382 | GRFYSLWDT | 1000.000 |
| | | 599 | ARVMSWWDY | 1000.000 |
| | | 288 | KRVYIAYST | 600.000 |
| | | 803 | KRKRGYIKN | 600.000 |
| | | 649 | MRTLDVDYV | 600.000 |
| | | 592 | RQNTDEHAR | 300.000 |
| | | 346 | KQEFQTLFF | 300.000 |
| | | 230 | LQFTYYLWV | 300.000 |
| | | 189 | TRELWNQGA | 200.000 |
| | | 108 | YRSTHHLAS | 200.000 |
| | | 785 | PRVTNIFPK | 200.000 |
| | | 616 | NRTTLVDNN | 200.000 |
| | | 316 | IRTSEHMAA | 200.000 |
| | | 166 | IRDVCVFLA | 200.000 |
| | | 591 | LRQNTDEHA | 200.000 |
| | | 63 | SQPAGWQSL | 200.000 |
| | | 351 | TLFFLGVSL | 150.000 |
| | | 347 | QEFQTLFFL | 150.000 |
| | | 386 | SLWDTGYAK | 150.000 |
| | | 716 | LMYKMSYYR | 125.000 |
| | | 609 | YQIAGMANR | 100.000 |
| | | 406 | HQPTTWVSF | 100.000 |
| | | 93 | IRFESIIHE | 100.000 |
| | | 106 | FNYRSTHHL | 100.000 |
| | | 128 | ERAWYPLGR | 100.000 |
| | | 723 | YRFGEMQLD | 100.000 |
| | | 331 | LQAYAFLQY | 100.000 |
| | 10 | 504 | KRNQGNLYDK | 6000.000 |
| | | 723 | YRFGEMQLDF | 5000.000 |
| | | 93 | IRFESIIHEF | 5000.000 |
| | | 288 | KRVYIAYSTF | 3000.000 |
| | | 679 | VRIAEGEHPK | 2000.000 |
| | | 517 | VRKHATEQEK | 2000.000 |
| | | 649 | MRTLDVDYVL | 2000.000 |
| | | 803 | KRKRGYIKNK | 1800.000 |
| | | 337 | LQYLRDRLTK | 1000.000 |
| | | 284 | QRYSKRVYIA | 1000.000 |
| | | 591 | LRQNTDEHAR | 1000.000 |
| | | 340 | LRDRLTKQEF | 1000.000 |
| | | 230 | LQFTYYLWVK | 1000.000 |
| | | 346 | KQEFQTLFFL | 600.000 |
| | | 458 | VRLMLTLTPV | 600.000 |
| | | 489 | KRENPPVEDS | 600.000 |
| | | 805 | KRGYIKNKLV | 540.000 |
| | | 777 | NRETLDHKPR | 300.000 |
| | | 213 | SRSVAGSFDN | 200.000 |
| | | 68 | WQSLLSFTIL | 200.000 |
| | | 108 | YRSTHHLASH | 200.000 |
| | | 331 | LQAYAFLQYL | 200.000 |
| B_2705 | 10 | 616 | NRTTLVDNNT | 200.000 |
| | | 29 | SRHGHHGPGA | 200.000 |
| | | 316 | IRTSEHMAAA | 200.000 |
| | | 702 | FRVDKAGSPT | 200.000 |
| | | 732 | FRTPPGFDRT | 200.000 |
| | | 63 | SQPAGWQSLL | 200.000 |
| | | 592 | RQNTDEHARV | 180.000 |
| | | 716 | LMYKMSYYRF | 125.000 |
| | | 406 | HQPTTWVSFF | 100.000 |
| | | 382 | GRFYSLWDTG | 100.000 |
| B_3501 | 10 | 686 | HPKDIRESDY | 240.000 |
| B_3701 | 10 | 704 | VDKAGSPTLL | 200.000 |
| B_3801 | 9 | 573 | NHDGTRNIL | 180.000 |
| B_3901 | 9 | 573 | NHDGTRNIL | 135.000 |
| | 10 | 164 | VHIRDVCVFL | 180.000 |
| B_4403 | 9 | 438 | DERVFVALY | 1080.000 |
| | | 762 | SEHWLVRIY | 720.000 |
| | | 100 | HEFDPWFNY | 180.000 |
| | | 596 | DEHARVMSW | 108.000 |
| | 10 | 744 | AEIGNKDIKF | 1350.000 |
| | | 319 | SEHMAAAGVF | 180.000 |
| B_5101 | 9 | 308 | IPFVGFQPI | 1384.240 |
| | | 425 | FPAGLWFCI | 572.000 |
| | | 261 | SAWGGYVFI | 484.000 |
| | | 90 | FAVIRFESI | 314.600 |
| | | 208 | VPGYISRSV | 314.600 |
| | | 392 | YAKIHIPII | 314.600 |
| | | 743 | NAEIGNKDI | 292.820 |
| | | 292 | IAYSTFYIV | 286.000 |
| | | 18 | SPWSGLMAL | 242.000 |
| | | 560 | NAYSSPSVV | 220.000 |
| | | 129 | RAWYPLGRI | 220.000 |
| | | 758 | EAFTSEHWL | 220.000 |
| | | 443 | VALYAISAV | 157.300 |
| | | 644 | AAYKIMRTL | 146.410 |
| | Mers | Position | Sequence | Score |
| | | 273 | IPLHVFVLL | 143.000 |
| | | 200 | LAACFIAIV | 143.000 |
| | | 64 | QPAGWQSLL | 121.000 |
| | | 332 | QAYAFLQYL | 121.000 |
| | | 300 | VGLILSMQI | 114.400 |
| | | 54 | APAGLSGGL | 110.000 |
| | | 360 | AAGAVFLSV | 110.000 |
| | 10 | 465 | TPVVCMLSAI | 484.000 |
| | | 174 | APTFSGLTSI | 484.000 |
| | | 261 | SAWGGYVFII | 440.000 |
| | | 758 | EAFTSEHWLV | 400.000 |
| | | 216 | VAGSFDNEGI | 314.600 |
| | | 681 | IAEGEHPKDI | 314.600 |
| B_5101 | 10 | 90 | FAVIRFESII | 286.000 |
| | | 360 | AAGAVFLSVI | 220.000 |
| | | 196 | GAGLLAACFI | 220.000 |
| | | 264 | GGYVFIINLI | 212.960 |
| | | 529 | EGLGPNIKSI | 212.960 |
| | | 378 | APWSGRFYSL | 200.000 |
| | | 390 | TGYAKIHIPI | 176.000 |
| | | 359 | LAAGAVFLSV | 157.300 |
| | | 143 | YPGLMITAGL | 143.000 |
| | | 273 | IPLHVFVLLL | 130.000 |
| | | 49 | APPKPAPAGL | 121.000 |
| | | 6 | APESKHKSSL | 110.000 |
| | | 129 | RAWYPLGRIV | 110.000 |
| | | 449 | SAVYFAGVMV | 110.000 |
| | | 560 | NAYSSPSVVL | 100.000 |
| B_5102 | 9 | 308 | IPFVGFQPI | 2420.000 |
| | | 129 | RAWYPLGRI | 2000.000 |
| | | 90 | FAVIRFESI | 1320.000 |
| | | 261 | SAWGGYVFI | 1210.000 |
| | | 425 | FPAGLWFCI | 880.000 |
| | | 292 | IAYSTFYIV | 550.000 |
| | | 18 | SPWSGLMAL | 550.000 |
| | | 560 | NAYSSPSVV | 500.000 |
| | | 228 | FALQFTYYL | 399.300 |
| | | 273 | IPLHVFVLL | 363.000 |
| | | 644 | AAYKIMRTL | 332.750 |
| | | 443 | VALYAISAV | 330.000 |
| | | 332 | QAYAFLQYL | 302.500 |
| | | 758 | EAFTSEHWL | 275.000 |
| | | 197 | AGLLAACFI | 264.000 |
| | | 806 | RGYIKNKLV | 242.000 |
| | | 300 | VGLILSMQI | 240.000 |
| | | 392 | YAKIHIPII | 220.000 |
| | | 208 | VPGYISRSV | 220.000 |
| | | 743 | NAEIGNKDI | 133.100 |
| | | 64 | QPAGWQSLL | 121.000 |
| | | 314 | QPIRTSEHM | 119.790 |
| | | 200 | LAACFIAIV | 110.000 |
| | | 54 | APAGLSGGL | 110.000 |
| | | 360 | AAGAVFLSV | 110.000 |
| | | 264 | GGYVFIINL | 110.000 |
| | 10 | 90 | FAVIRFESII | 1200.000 |
| | | 465 | TPVVCMLSAI | 1200.000 |
| | | 261 | SAWGGYVFII | 1100.000 |
| | | 129 | RAWYPLGRIV | 550.000 |
| | | 758 | EAFTSEHWLV | 550.000 |
| | | 378 | APWSGRFYSL | 500.000 |
| | | 264 | GGYVFIINLI | 440.000 |
| | | 174 | APTFSGLTSI | 440.000 |
| | | 390 | TGYAKIHIPI | 400.000 |
| | | 529 | EGLGPNIKSI | 351.384 |
| | | 328 | FALLQAYAFL | 330.000 |
| | | 273 | IPLHVFVLLL | 330.000 |
| | | 449 | SAVYFAGVMV | 300.000 |
| B_5201 | 10 | 427 | AGLWFCIKNI | 290.400 |
| | | 560 | NAYSSPSVVL | 250.000 |
| | | 216 | VAGSFDNEGI | 242.000 |
| | | 143 | YPGLMITAGL | 242.000 |
| | | 196 | GAGLLAACFI | 220.000 |
| | | 360 | AAGAVFLSVI | 200.000 |
| | | 83 | AGFSSRLFAV | 200.000 |
| | | 362 | GAVFLSVIYL | 165.000 |
| | | 681 | IAEGEHPKDI | 121.000 |
| | | 359 | LAAGAVFLSV | 121.000 |
| | | 355 | LGVSLAAGAV | 120.000 |
| | | 453 | FAGVMVRLML | 110.000 |
| | | 49 | APPKPAPAGL | 110.000 |
| B_5103 | 9 | 560 | NAYSSPSVV | 300.000 |
| | | 292 | IAYSTFYIV | 300.000 |
| | | 443 | VALYAISAV | 159.720 |
| | | 261 | SAWGGYVFI | 133.100 |
| | | 806 | RGYIKNKLV | 120.000 |
| | | 90 | FAVIRFESI | 110.000 |
| | | 200 | LAACFIAIV | 110.000 |
| | | 360 | AAGAVFLSV | 110.000 |
| | | 743 | NAEIGNKDI | 110.000 |
| | | 392 | YAKIHIPII | 110.000 |
| | | 129 | RAWYPLGRI | 100.000 |
| | 10 | 264 | GGYVFIINLI | 145.200 |
| | | 758 | EAFTSEHWLV | 132.000 |
| | | 390 | TGYAKIHIPI | 132.000 |
| | | 449 | SAVYFAGVMV | 121.000 |
| | | 359 | LAAGAVFLSV | 121.000 |
| | | 196 | GAGLLAACFI | 121.000 |
| | | 216 | VAGSFDNEGI | 110.000 |
| | | 681 | IAEGEHPKDI | 110.000 |
| | | 261 | SAWGGYVFII | 110.000 |
| | | 129 | RAWYPLGRIV | 100.000 |
| | | 90 | FAVIRFESII | 100.000 |
| | | 360 | AAGAVFLSVI | 100.000 |
| B_5201 | 9 | 531 | LGPNIKSIV | 330.000 |
| | | 292 | IAYSTFYIV | 123.750 |
| | | 130 | AWYPLGRIV | 120.000 |
| | 10 | 806 | RGYIKNKLVF | 165.000 |
| | | 129 | RAWYPLGRIV | 100.000 |
| B_5801 | 9 | 239 | KSVKTGSVF | 240.000 |
| | | 12 | KSSLNSSPW | 240.000 |
| | | 380 | WSGRFYSLW | 120.000 |
| | 10 | 239 | KSVKTGSVFW | 480.000 |
| | | 617 | RTTLVDNNTW | 290.400 |
| | | 72 | LSFTILFLAW | 158.400 |
| | | 254 | LSYFYMVSAW | 144.000 |
| B60 | 9 | 347 | QEFQTLFFL | 160.000 |
| | | 222 | NEGIAIFAL | 160.000 |
| | 10 | 757 | EEAFTSEHWL | 320.000 |
| | | 190 | RELWNQGAGL | 320.000 |
| | | 522 | TEQEKTEEGL | 160.000 |
| B62 | 9 | 283 | MQRYSKRVY | 132.000 |
| | | 365 | FLSVIYLTY | 105.600 |

ii) SIMP Homology of with Other Genes and Proteins

As mentioned previously, the cloning of hSIMP was carried out starting with the putative amino acid sequence of a mouse minor histocompatibility antigen (MiHA) called “B6^{dom1}”. Prior to the present invention, the identity of the mouse gene encoding the B6^{dom1} MiHA was unknown. A blast search revealed that human SIMP is highly homologous to a mouse gene (GENBANK™ accession No AK018758) for which no formal name nor biological role have been assigned. This mouse gene, referred hereinafter as mouse SIMP (mSIMP), contains an open reading frame of 2469 bp (SEQ. ID. NO: 3) and encodes a protein of some 823 amino acids (SEQ. ID. NO: 4).

Although not shown, the cDNA sequence of SEQ ID NO:150 of international PCT application WO 01/19988 (see GENBANK™ accession No AK027789) shares 100% identity with nucleic acids no 1510 to 2481 of hSIMP. The protein sequence of SEQ ID NO:151 of the same PCT application (see GENBANK™ accession No BAB55370) shares 100% identity with the C-terminal end of the human SIMP protein (amino acids no 541 to 826). SEQ ID NO:150 and 151 of WO 01/19988 correspond to an EST and a predicted protein for which no function is described.

Analysis of human and mouse SIMPs confirms that the two genes and proteins are highly homologous to each other. Indeed, the conservation between the hSIMP and mSIMP genes is striking. These are roughly 90% identical at the DNA level, while in terms of encoded amino acids the two proteins are 97% identical. This is strongly suggestive of the existence of a strong selection pressure to maintain the sequence and biological function of this protein across species. Since mSIMP is ubiquitously expressed in mice, it is expected that the same holds true for hSIMP. Applicants preliminary results (arrays) show that SIMP is fairly ubiquitous in human (not shown). However, sequencing of hSIMP cDNA in fourteen unrelated individuals (not shown) confirms that contrary to mSIMP, hSIMP is not polymorphic, i.e. hSIMP occurs in a single form in human. This means that probes and reagents that recognize or react with hSIMP from one individual should recognize or react in the same way with hSIMP from all human subjects.

Blast searches were also made to identify sequence identity between hSIMP, mSIMP and other existing sequences. As shown hereafter in Table 2 and Table 3, hSIMP and mSIMP were found to be highly homologous to yeast STT3 (GENBANK™ accession No D28952 (DNA; SEQ ID NO:5) and No BM06079 (protein; SEQ ID NO:6); T12A2.2 C. Elegans (GENBANK™ accession No P46975 (protein; SEQ ID NO:13); drosophila STT3 (GENBANK™ No AF132552 (DNA; SEQ ID NO:7 and protein; SEQ ID NO:8), mouse ITM1 (GENBANK™ accession No NM_{—}008408 (DNA; SEQ ID NO:9) and NP_{—}032434 (protein; SEQ ID NO:10)), and human ITM1 (GENBANK™ accession No NM_{—}002219 (DNA; SEQ ID NO:11) and No NP_{—}002210 (protein; SEQ ID NO:12)).

Standard techniques, such as the polymerase chain reaction (PCR) and DNA hybridization, may be used to clone additional SIMP homologues in other species.

TABLE 2
Comparison between human SIMP cDNA sequence and known nucleotide sequences*.

| | STT3 yeast | STT3 drosophila | ITM1 mouse | SIMP mouse | ITM1 human | SIMP human |
|---|---|---|---|---|---|---|
| | (SEQ ID NO: 5) | (SEQ ID NO: 7) | (SEQ ID NO: 9) | (SEQ ID NO: 3) | (SEQ ID NO: 11) | (SEQ ID NO: 1) |
| STT3 yeast | — | 58.6 | 57.8 | 54.9 | 58.2 | 54.8 |
| (SEQ ID NO: 5) | | | | | | |
| STT3 drosophila | 58.4 | — | 57.7 | 63 | 58 | 62.8 |
| (SEQ ID NO: 7) | | | | | | |
| ITM1 mouse | 57.7 | 57.4 | — | 56 | 92.3 | 55.5 |
| (SEQ ID NO: 9) | | | | | | |
| SIMP mouse | 54.7 | 63 | 56.2 | — | 55.7 | 90.3 |
| (SEQ ID NO: 3) | | | | | | |
| ITM1 human | 58.3 | 57.8 | 92.3 | 55.8 | — | 54.9 |
| (SEQ ID NO: 11) | | | | | | |
| SIMP human | 55 | 62.7 | 55.6 | 90.3 | 54.8 | — |
| (SEQ ID NO: 1) | | | | | | |

*Results are shown as percentage of identity

TABLE 3
Comparison between human SIMP amino acid sequence and known amino acid sequences.

| | STT3 yeast | T12A2.2 | | | | | |
|---|---|---|---|---|---|---|---|
| | (SEQ ID NO: | C. elegans | STT3 drosophila | ITM1 mouse | SIMP mouse | ITM1 human | SIMP human |
| | 6) | SEQ ID NO: 13 | (SEQ ID NO: 8) | (SEQ ID NO: 10) | (SEQ ID NO: 4) | (SEQ ID NO: 12) | (SEQ ID NO: 2) |
| STT3 yeast | — | 54/69 | 52/67 | 54/69 | 53/68 | 54/69 | 53/69 |
| (SEQ ID NO: 6) | | | | | | | |
| T12A2.2 | 54/69 | — | 65/78 | 56/71 | 66/79 | 56/71 | 66/78 |
| C. elegans | | | | | | | |
| (SEQ ID NO: 13) | | | | | | | |
| STT3 drosophila | 52/67 | 65/78 | — | 57/72 | 71/82 | 57/72 | 72/83 |
| (SEQ ID NO: 8) | | | | | | | |
| ITM1 mouse | 54/69 | 56/71 | 57/72 | — | 59/73 | 98/98 | 60/74 |
| (SEQ ID NO: 10) | | | | | | | |
| SIMP mouse | 53/68 | 66/79 | 71/82 | 59/73 | — | 59/73 | 97/97 |
| (SEQ ID NO: 4) | | | | | | | |
| ITM1 human | 54/69 | 56/71 | 57/72 | 98/98 | 59/73 | — | 59/73 |
| (SEQ ID NO: 12) | | | | | | | |
| SIMP human | 53/69 | 66/78 | 72/83 | 60/74 | 97/97 | 59/73 | — |
| (SEQ ID NO: 2) | | | | | | | |

* Results are shown as percentage of identity/homology

Interestingly, the hSIMP gene encodes a protein of 826 amino acids which exhibits 53% identity and 69% similarity to yeast STT3, which establishes it as a novel member of this group of genes. Yeast STT3 is a subunit of a large complex required for the appropriate co-translational N-glycosylation of proteins, a modification that is characteristic of eukaryotes and is involved in chaperone-mediated protein folding. Disruption of this gene in yeast demonstrated that it is essential for cell growth, underscoring its likelihood to be critical for normal cellular function in higher eukaryotes. There appears to be a family of proteins directly related to STT3, with homologs found even in lower organisms such as archaebacteria, in addition to equivalents in higher organisms including mice and humans. That these proteins are remarkably well conserved across divergent species indicates a strong evolutionary pressure for maintenance of biological function of this family.

The genes of mice and humans heretofore identified as being structurally and functionally related to STT3, is known as ITM1, for Integral Membrane Protein-1. The protein encoded by mouse ITM1 was found to contain many putative transmembrane domains and possesses roughly 52% identity and 66% similarity to yeast STT3, respectively. The T12A2.2 gene in C. elegans encodes a protein that is similarly conserved with both STT3 and ITM1, and represents another member of this family of proteins. In Drosophila melangoster there are homologs of both STT3 and ITM1 on different chromosomes, indicatory of the evolutionary separation of these genes. A human equivalent of ITM1 has also been cloned which has a similar degree of homology with STT3 as the mouse protein, but, interestingly, the proteins mice and humans are 97% identical, underlining the potentially major role of this protein in higher organisms.

Human SIMP is in turn 59% identical and 73% similar to human ITM1, which, while significant, distinguishes it from its human homolog. Intriguingly, hSIMP protein is more similar to the C. elegans and D. melangoster STT3-like proteins (roughly 70% identity and 80% similarity) than it is to human ITM1. This would suggest that hSIMP evolved separately from ITM1, and that indeed hSIMP and ITM1 are functionally distinct. This is further emphasized by the degree of homology between human and mouse ITM1; these two proteins are roughly 98% identical. Given the levels of identity between human SIMP and human ITM1, these two proteins presumably perform perhaps related but unique roles in humans. It is also proposed herein that the two genes are paralogs (i.e. homologous genes that diverged by gene duplication). Because hSIMP and hITM1 are paralogs, they may have similar roles, perhaps in different cell types. Accordingly, hSIMP may have a biological function similar to that of ITM1, and ITM1 an immunological function similar to that of hSIMP. For instance, we have verified using the BIMAS search tool, that similar to hSIMP, human ITM1 has the potential to generate protein fragments that bind with high affinity to HLA molecules (data not shown). The present invention therefore encompasses any use of such ITM1-derived polypeptides, particularly in cancer immunotherapy. The invention also encompasses any sequences, probe, kit, method involving human ITM1 for similar uses as those mentioned throughout the present application for human SIMP.

Given the high sequence homology of SIMP with STT3 and ITM1, it is reasonable to hypothesize that these proteins may have similar biological functions. Yeast STT3 and mouse ITM1 are known to be part of the oligosaccharyltransferase (OST) complex. N-linked protein glycosylation is an essential process in eukaryotic cells. In the central reaction, OST catalyzes the transfer of the oligosaccharide Glc₃MangGlcNac₂ from dolicholpyrophosphate onto asparagine residues of nascent polypeptide chains in the lumen of the endoplasmic reticulum. A major function for sugars is to contribute to the stability of the proteins to which they are attached. Moreover, specific glycoforms are involved in recognition events. Like protein translocation, N-linked glycosylation clearly belongs to the functions that the ER has inherited from the prokaryotic, most likely archaeal, plasma membrane. STT3 and ITM1 proteins, transmembrane proteins with a C-terminal, lumenally oriented, hydrophilic domain, are part of the OST complex. Depletion of STT3 protein and mutation of STT3 result in loss of transferase activity in vivo, a deficiency in the assembly of the OST complex and loss of cell growth and viability which may be corrected by transfection with STT3 or ITM1. Consistent with a role of STT3p homologs in cell proliferation, ITM1 transcripts are expressed predominantly in tissues undergoing active proliferation and differentiation. Tables 1 and 2 also shows a surprising degree of conservation of the STT3 protein between yeast and higher eukaryotes.

Furthermore, OST activity seems to be particularly important for the cells of the immune system. This might not be surprising since almost all of the key molecules involved in the innate and adaptive immune response are glycoproteins. Specific glycoforms control crucial events in recognition of APCs by T-cells: assembly of MHC-peptide complexes, formation of immunological synapse, recognition of antigenic peptide-loaded MHC molecules by the TCRs and signal transduction. In previous studies OST activity was found to increase 10-fold after mitogen activation of PBLs. The number of copies of B6^{dom1} MiHA per cell (a peptide from mSIMP) was shown to increase by 128-fold on mitogen activated T-cells relative to resting splenocytes. Interestingly, previous studies have shown levels of Dad1 (the defender against apoptotic cell death, a member of the OST complex) are modulated during T-cell development, to reach maximal expression in mature T-cells, and peripheral T-cells of Dadl-transgenic mice display hyperproliferation in response to stimuli. All these observations suggest that SIMP could be particularly important for cells with a high proliferation rate.

iii) T-Cell Immunotherapy Targeted to MHC-Associated Peptides Encoded by SIMP

SIMP polypeptides may be useful for eliminating tumoral cells in human and more particularly hematopoietic cancer cells. This may be achieved by injecting into a cancer bearing host T-lymphocytes, that recognize complexes of SIMP-derived peptide/MHC on cancer cells. In a preferred embodiment, the SIMP-derived peptide comprises at least eight sequential amino acids of SEQ ID NO:2 (hSIMP). More preferably, the fragment is selected from the fragment listed in Table 1.

Since ITM1 and SIMP are paralogs, the method could potentially be used by targeting ITM1-derived peptides/MHC complexes as well. Preferably, the ITM1-derived peptide will be selected from the peptides that comprise at least nine sequential amino acids of SEQ ID NO: 12 (hITM1).

Some of the methods of T-lymphocytes selection and methods of immunotherapy are described in detail in PCT application No. PCT/CA01/01477 which is incorporated herein by reference. Four immunotherapeutic situations can be envisaged depending on the type of effector T-cells used and on the nature of the target SIMP-derived peptide. Indeed, T-cells can be i) allogeneic, that is, T-cells obtained from another individual or ii) self, that is, the patient's T-cells. The target SIMP peptide can be either polymorphic or non polymorphic.

Situation 1: Allogeneic T-Cells, Non Polymorphic Peptide Target.

According to a preferred embodiment, T-cells that specifically recognize the target MHC/SIMP peptide epitope (allo MHC-restricted T-cells) will be generated from an MHC-incompatible donor. In vitro T-cell expansion will be carried out using current cell culture techniques following stimulation with the target epitope or a heteroclitic variant of the SIMP peptide (a variant of the peptide whose sequence has been modified to increase its immunogenicity). Heteroclitic peptides may be synthesized by replacing one (or a few) natural amino acids in a polypeptide by an amino acid that is predicted (using a tool such as BIMAS HLA peptide binding predictions) to bind with a superior affinity to a few MHC molecules. T-cells that react with the target epitope will be purified with the MHC/SIMP-peptide tetramers, cloned, and their innocuity for normal host cells will be assessed with in vitro assays (³H-thymidine or ⁵¹Cr release, cytokine production). The selected and expanded T-cell clones will be injected into the blood vessels of the recipient. Injected T lymphocytes will then “seek and destroy” neoplastic cells located in various tissues and organs.

Situation 2: Allogeneic T-Cells, Polymorphic Peptide Target

This embodiment is carried out as in Situation 1, except that the donor that is selected is MHC-identical with the recipient. MHC identity is assessed based on currently available methods of MHC typing using antibodies and nucleotide probes. In this case, the T-cells are said to be self MHC-restricted and the target peptide is called an MiHA.

Situation 3: Self T-Cells Transfected with an Allogeneic TCR Specific for a Polymorphic or Non Polymorphic Peptide Target

T-cell clones are generated as in Situations 1 and 2. However, rather than injecting allogeneic T-cells into the recipient, the T-cell receptor (TCR) of these allogeneic T-cells is cloned and used to transfect recipient T-cells in vitro (Stanislawski et al., 2001, Nat. Immunol 2:962-970; Kessels et al., 2001, Nat. Immunol 2:957-961). Transfected T-cells are then injected back into the recipient as described previously.

Situation 4: Self T-Cells Not Transfected with an Allogeneic TCR and Targeted to a Polymorphic or Non Polymorphic Target

According to a preferred embodiment, T-cells from a cancer bearing patient are stimulated in vitro with antigen presenting cells expressing the target MHC-associated SIMP-peptide or a heteroclitic variant of the SIMP peptide (See situation 1). Expression of the target peptide can be either endogenous, or induced by RNA or cDNA transfection or pulsing with synthetic peptide using currently available methods. T-cells reacting with optimal avidity with cells expressing the target epitope are purified and expanded using currently available methods (Yee et al., 1999, J. Immunol. 162:2227-2234; Bullock et al., 2001, J. Immunol. 167:5824-5831) then injected into the recipients.

iv) SIMP Therapies

Therapies may be designed to circumvent or overcome an inadequate SIMP gene expression. Indeed, SIMP seems to be expressed in higher levels in high proliferative cells. Therefore, SIMP protein or polypeptides may be effective proliferative agents and increasing their intracellular levels may help or stimulate cell proliferation. This could be accomplished for instance by transfection of SIMP cDNA. Thus, cancer treatment with radiotherapy and chemotherapy is currently limited by the hematological toxicity of these treatment modalities, that is, the length of time required for proliferation of hematopoietic progenitors to restore normal levels of blood cells. Therefore, the following strategy could be used to shorten the length of blood cytopenias following chemo or radiotherapy: hematopoietic progenitors harvested from the blood or the bone marrow of a patient are transfected with SIMP cDNA and the transfected cells are then re-injected into the patient before a cycle of chemo/radiotherapy.

To obtain large amounts of pure SIMP, cultured cell systems would be preferred. Delivery of the protein to the affected tissues can then be accomplished using appropriate packaging or administrating systems. Alternatively, it is conceivable that small molecule analogs could be used and administered to act as SIMP agonists and in this manner produce a desired physiological effect. Methods for finding such molecules are provided herein.

v) Downregulation of SIMP Expression

1) For Cancer Therapy

We have previously shown that T-cells targeted to the B6^{dom1} peptide (derived from mSIMP) were extremely effective in eradicating B6^{dom1}-positive cells (see PCT/CA01/01477). A corollary is that cancer cells could not escape a T-cell attack by downregulating SIMP expression or by expressing SIMP mutants. Thus, consistent with a crucial role of STT3 homologs in cell proliferation, we propose that SIMP expression is essential for cancer cell proliferation. Accordingly, downmodulation of SIMP could be used to treat cancer. Therefore, the invention relates to methods for modulating tumoral cell survival or for eliminating a tumoral cell in a human by reducing cellular expression levels of a human SIMP polypeptide. In a preferred embodiment, this is achieved by delivering an antisense into the tumoral cells. This can be achieved by intravenous injection using currently available methods (e.g. Crooke et al, (2000), Oncogene 19, 6651-6659; Stein et al., (2001), J. Clin. Invest 108, 641-644; and Tamm et al., (2001), Lancet 358, 489-497. Theoretically, this approach could be used for all types of cancer and should be most useful for those that proliferate more rapidly, that is, the most malignant cancers (e.g. hematopoietic cancer, lung cancers, intestine cancers, prostate cancer, testis cancer, breast cancer, melanomas, pancreatic cancer sarcomas, prostate cancer and hematologic cancers).

2) For Modulating Immune Responses

As mentioned above, OST activity seems to be particularly important for T-lymphocytes function. Furthermore, the previous observation that the number of copies of B6^{dom1} MiHA per cell (a peptide from mSIMP) was increased 128-fold on mitogen activated T-cells relative to resting splenocytes, suggests that SIMP is very important for T-cell activation/proliferation. Accordingly, downmodulation of SIMP expression could be used to dampen immune responses, particularly in the context of transplantation or autoimmune diseases.

Therefore, the invention also relates to methods for modulating an immune response by reducing cellular expression levels of a SIMP polypeptide. In a preferred embodiment, the method is used for decreasing lymphoid cell proliferation, and it comprises the step of decreasing in these cells cellular expression levels of a SIMP polypeptide. Such a method may be particularly useful for dampening deleterious immune responses occurring in recipients of organ or tissue transplant and in people with autoimmune disease. We infer that inhibition of SIMP function could be useful to prevent or treat transplant rejection and to treat autoimmune diseases such as diabetes, multiple sclerosis, rheumatoid arthritis etc. Preferably, reduced SIMP cellular expression is obtained by delivering a SIMP antisense into lymphoid cells by intravenous injection.

According to a related aspect of the two above-mentioned methods, the invention relates to antisense nucleic acids and to pharmaceutical compositions comprising such antisenses, the antisense being capable of reducing hSIMP cellular levels of expression. Preferably, the antisense nucleic acid is complementary to a nucleic acid sequence encoding a hSIMP protein or encoding any of the polypeptides derived therefrom and more particularly those listed in Table 1. More preferably, the antisense hybridizes under high stringency conditions to a genomic sequence or to a mRNA. Even more preferably, the antisense of the invention hybridizes under high stringency conditions to SEQ ID NO: 1 (hSIMP) or to a complementary sequence thereof. A non limitative example of high stringency conditions includes:

a) pre-hybridization and hybridization at 68° C. in a solution of 5×SSPE (1×SSPE=0.18 M NaCl, 10 mM NaH₂PO₄); 5× Denhardt solution; 0.05% (w/v) sodium dodecyl sulfate (SDS); et 100 μg/ml salmon sperm DNA;

b) two washings for 10 min at room temperature with 2×SSPE and 0.1% SDS;

c) one washing at 60° C. for 15 min with 1×SSPE and 0.1% SDS; and

d) one washing at 60° C. for 15 min with 0.1×SSPE et 0.1% SDS.

vi) Administration of SIMP Polypeptides, Modulators of SIMP Synthesis or Function

A SIMP protein, polypeptide, or modulator (e.g. antisense) may be administered within a pharmaceutically acceptable diluent, carrier, or excipient, in unit dosage form. Conventional pharmaceutical practice may be used to provide suitable formulations or compositions to administer SIMP protein, polypeptide, or modulator to patients. Administration may begin before the patient is symptomatic. Any appropriate route of administration may be employed, for example, administration may be parenteral, intravenous, intraarterial, subcutaneous, intramuscular, intracranial, intraorbital, ophthalmic, intraventricular, intracapsular, intraspinal, intracisternal, intraperitoneal, intranasal, aerosol, by suppositories, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington's Pharmaceutical Sciences.” Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If desired, treatment with a SIMP protein, polypeptide, or modulatory compound may be combined with more traditional therapies for the disease such as surgery, steroid therapy, or chemotherapy for autoimmune disease; other immunosuppressive agents for transplant rejection; and radiotherapy, chemotherapy for cancer.

According to a preferred embodiment, A SIMP antisense would be incorporated in a pharmaceutical composition comprising at least one of the oligonucleotides defined previously, and a pharmaceutically acceptable carrier. The amount of antisense present in the composition of the present invention is a therapeutically effective amount. A therapeutically effective amount of antisense is that amount necessary so that the antisense performs its biological function without causing overly negative effects in the host to which the composition is administered. The exact amount of oligonucleotides to be used and composition to be administered will vary according to factors such as the oligo biological activity, the type of condition being treated, the mode of administration, as well as the other ingredients in the composition. Typically, the composition will be composed of about 1% to about 90% of antisense, and about 20 μg to about 20 mg of antisense will be administered. For preparing and administering antisenses as well as pharmaceutical compositions comprising the same, methods well known in the art may be used. For instance, see Crooke et al. (Oncogene, 2000, 19:6651-6659) and Tamm et al. (Lancet 200, 1358:489-497) for a review of antisense technology in cancer chemotherapy.

vii) Upregulation of SIMP Expression

Upregulation of SIMP expression in T-lymphocytes could be used to increase T-lymphocyte proliferation following antigen encounter. Indeed, it is suggested that upregulation of SIMP would increase the size of effector T-cell and memory T-cell pools, that is, the efficacy of T-cell responses and the duration of a biologically relevant (protective) T-cell memory. In other words, increased SIMP function would be used as an immune adjuvant.

Therefore, the invention also relates to methods for modulating an immune response by increasing cellular expression levels of a SIMP polypeptide in lymphoid cells. In a preferred embodiment, such a method is used for increasing the level and/or the duration of an antigen-primed lymphocyte proliferation. Preferably, this is achieved by transfecting in vivo or ex vivo lymphocytes with a SIMP cDNA. Targeted lymphocytes can be CD4 T-cells and/or CD8 T-cells and/or B-cells.

viii) Synthesis of SIMP and Fragments Thereof

The characteristics of the cloned SIMP gene sequence may be analyzed by introducing the sequence into various cell types or using in vitro extracellular systems. The function of SIMP may then be examined under different physiological conditions. The SIMP DNA sequence may be manipulated in studies to understand the expression of the gene and gene product. Alternatively, cell lines may be produced which overexpress the gene product allowing purification of SIMP for biochemical characterization, large-scale production, antibody production, and patient therapy.

For protein expression, eukaryotic and prokaryotic expression systems may be generated in which the SIMP gene sequence is introduced into a plasmid or other vector which is then introduced into living cells. Constructs in which the SIMP cDNA sequence containing the entire open reading frame inserted in the correct orientation into an expression plasmid may be used for protein expression. Alternatively, portions of the sequence, including wild-type or mutant SIMP sequences, may be inserted. Prokaryotic and eukaryotic expression systems allow various important functional domains of the protein to be recovered as fusion proteins and then used for binding, structural and functional studies and also for the generation of appropriate antibodies.

Eukaryotic expression systems permit appropriate post-translational modifications to expressed proteins. This allows for studies of the SIMP gene and gene product including determination of proper expression and post-translational modifications for biological activity, identifying regulatory elements located in the 5′ region of the SIMP gene and their role in tissue regulation of protein expression. It also permits the production of large amounts of normal and mutant proteins for isolation and purification, to use cells expressing SIMP as a functional assay system for antibodies generated against the protein, to test the effectiveness of pharmacological agents or as a component of a signal transduction system, to study the function of the normal complete protein, specific portions of the protein, or of naturally occurring polymorphisms and artificially produced mutated proteins. The SIMP DNA sequence may be altered by using procedures such as restriction enzyme digestion, DNA polymerase fill-in, exonuclease deletion, terminal deoxynucleotide transferase extension, ligation of synthetic or cloned DNA sequences and site directed sequence alteration using specific oligonucleotides together with PCR.

A SIMP polypeptide may be produced by a stably-transfected mammalian cell line. A number of vectors suitable for stable transfection of mammalian cells are available to the public, as are methods for constructing such cell lines.

Once the recombinant protein is expressed, it is isolated by, for example, affinity chromatography. In one example, an anti-SIMP antibody, which may be produced by the methods described herein, can be attached to a column and used to isolate the SIMP protein. Lysis and fractionation of SIMP-harboring cells prior to affinity chromatography may be performed by standard methods. Once isolated, the recombinant protein can, if desired, be purified further.

Methods and techniques for expressing recombinant proteins and foreign sequences in prokaryotes and eukaryotes are well known in the art and will not be described in more detail. One can refer, if necessary to Joseph Sambrook, David W. Russell, Joe Sambrook Molecular Cloning: A Laboratory Manual 2001 Cold Spring Harbor Laboratory Press. Those skilled in the art of molecular biology will understand that a wide variety of expression systems may be used to produce the recombinant protein. The precise host cell used is not critical to the invention. The SIMP protein may be produced in a prokaryotic host (e.g., E. coli) or in a eukaryotic host (e.g., S. cerevisiae, insect cells such as Sf21 cells, or mammalian cells such as COS-1, NIH 3T3, or HeLa cells). These cells are publicly available, for example, from the American Type Culture Collection, Rockville, Md. The method of transduction and the choice of expression vehicle will depend on the host system selected.

Polypeptides of the invention, particularly short SIMP fragments, may also be produced by chemical synthesis. These general techniques of polypeptide expression and purification can also be used to produce and isolate useful SIMP fragments or analogs, as described herein.

The polypeptides of the present invention may also be incorporated in polypeptides of various length, preferably from about 8 to about 50 amino acids, an more preferably from about 8 to about 12 amino acids. According to a preferred embodiment, the peptides are incorporated in a tetrameric complex comprising a plurality of identical or different SIMP peptides/polypeptides according to the invention. According to another preferred embodiment, the peptides of the invention are incorporated into a support comprising at least two peptidic molecules. Examples of suitable supports include polymers, lipidic vesicles, microsphere, latex beads, polystyrene beads, proteins and the like.

Skilled artisans will recognize that a mammalian SIMP, or a fragment thereof (as described herein), may serve as an active ingredient in a therapeutic composition. This composition, depending on the SIMP or fragment included, may be used to regulate cell proliferation, survival and apoptosis and thereby treat any condition that is caused by a disturbance in cell proliferation, accumulation or replacement. Thus, it will be understood that another aspect of the invention described herein, includes the compounds of the invention in a pharmaceutically acceptable carrier.

ix) SIMP Antibodies

The invention features a purified antibody (monoclonal and polyclonal) that specifically binds to a SIMP protein.

The antibodies of the invention may be prepared by a variety of methods using the SIMP proteins or polypeptides described above. For example, the SIMP polypeptide, or antigenic fragments thereof, may be administered to an animal in order to induce the production of polyclonal antibodies. Alternatively, antibodies used as described herein may be monoclonal antibodies, which are prepared using hybridoma technology (see, e.g., Hammerling et al., In Monoclonal Antibodies and T-Cell Hybridomas, Elsevier, NY, 1981). The invention features antibodies that specifically bind human or murine SIMP polypeptides, or fragments thereof. In particular, the invention features “neutralizing” antibodies. By “neutralizing” antibodies is meant antibodies that interfere with any of the biological activities of the SIMP polypeptide, particularly the ability of SIMP to inhibit apoptosis. The neutralizing antibody may reduce the ability of SIMP polypeptides to inhibit apoptosis by, preferably 50%, more preferably by 70%, and most preferably by 90% or more. Any standard assay of apoptosis, including those described herein, may be used to assess potentially neutralizing antibodies. Once produced, monoclonal and polyclonal antibodies are preferably tested for specific SIMP recognition by Western blot, immunoprecipitation analysis or any other suitable method.

In addition to intact monoclonal and polyclonal anti-SIMP antibodies, the invention features various genetically engineered antibodies, humanized antibodies, and antibody fragments, including F(ab′)₂, Fab′, Fab, Fv and sFv fragments. Antibodies can be humanized by methods known in the art. Fully human antibodies, such as those expressed in transgenic animals, are also features of the invention.

Antibodies that specifically recognize SIMP (or fragments of SIMP), such as those described herein, are considered useful to the invention. Such an antibody may be used in any standard immunodetection method for the detection, quantification, and purification of a SIMP polypeptide. Preferably, the antibody binds specifically to SIMP. The antibody may be a monoclonal or a polyclonal antibody and may be modified for diagnostic or for therapeutic purposes. The most preferable antibody binds the SIMP polypeptide sequences of SEQ. ID NO:1 (hSIMP) and/or SEQ. ID NO:4 (mSIMP).

The antibodies of the invention may, for example, be used in an immunoassay to monitor SIMP expression levels, to determine the subcellular location of a SIMP or SIMP fragment produced by a mammal or to determine the amount of SIMP or fragment thereof in a biological sample. Antibodies that inhibit SIMP described herein may be especially useful for conditions where decreased SIMP function would be advantageous that is, inhibition of cancer cell proliferation, prevention of rejection and the treatment of autoimmune disease. In addition, the antibodies may be coupled to compounds for diagnostic and/or therapeutic uses such as radionucleotides for imaging and therapy and liposomes for the targeting of compounds to a specific tissue location. The antibodies may also be labeled (e.g. immunofluorescence) for easier detection.

x) Assessment of SIMP Intracellular or Extracellular Levels

As noted, the antibodies described above may be used to monitor SIMP protein expression and/or to determine the amount of SIMP or fragment thereof in a biological sample.

In addition, in situ hybridization may be used to detect the expression of the SIMP gene. As it is well known in the art, in situ hybridization relies upon the hybridization of a specifically labeled nucleic acid probe to the cellular RNA in individual cells or tissues. Therefore, oligonucleotides or cloned nucleotide (RNA or DNA) fragments corresponding to unique portions of the SIMP gene may be used to asses SIMP cellular levels or detect specific mRNA species. Such an assessment may also be done in vitro using well known methods (Northern analysis, quantitative PCR, etc.)

Determination of the amount of SIMP or fragment thereof in a biological sample may be especially useful for diagnosing a cell proliferative disease or an increased likelihood of such a disease, particularly in a human subject, using a SIMP nucleic acid probe or SIMP antibody. Preferably the disease is a rapidly growing cancer or a cancer that displays a short doubling time (e.g. hematopoietic cancer, lung cancers, prostate cancer, testis cancer, breast cancer, melanomas, pancreatic cancer intestine cancers, sarcomas, prostate cancer and hematologic cancers). This may be achieved by contacting, in vitro or in vivo, a biological sample (such as a blood sample or a tissue biopsy) from an individual suspected of harboring cancer cells, with a SIMP antibody or a probe according to the invention, in order to evaluate the amount of SIMP in the sample or the cells therein. The measured amount would be indicative of the probability of the subject of having proliferating tumoral cells since it is expected that these cells have a higher level of SIMP expression.

In a related aspect, the invention features a method for detecting the expression of SIMP in tissues comprising, i) providing a tissue or cellular sample; ii) incubating said sample with an anti-SIMP polyclonal or monoclonal antibody; and iii) visualizing the distribution of SIMP.

Assay kits for determining the amount of SIMP in a sample would also be useful and are within the scope of the present invention. Such a kit would preferably comprise SIMP antibody(ies) or probe(s) according to the invention and at least one element selected from the group consisting of instructions for using the kit, assay tubes, enzymes, reagents or reaction buffer(s), enzyme(s).

xi) Identification of Molecules that Modulate SIMP Protein Expression

SIMP cDNAs may be used to facilitate the identification of molecules that increase or decrease SIMP expression. In one approach, candidate molecules are added, in varying concentration, to the culture medium of cells expressing SIMP mRNA. SIMP expression is then measured, for example, by Northern blot analysis using a SIMP cDNA, or cDNA or RNA fragment, as a hybridization probe. The level of SIMP expression in the presence of the candidate molecule is compared to the level of SIMP expression in the absence of the candidate molecule, all other factors (e.g. cell type and culture conditions) being equal.

Compounds that modulate the level of SIMP may be purified, or substantially purified, or may be one component of a mixture of compounds such as an extract or supernatant obtained from cells (Ausubel et al., supra). In an assay of a mixture of compounds, SIMP expression is tested against progressively smaller subsets of the compound pool (e.g., produced by standard purification techniques such as HPLC or FPLC) until a single compound or minimal number of effective compounds is demonstrated to modulate SIMP expression.

Compounds may also be screened for their ability to modulate SIMP-biological activity (e.g. enhancement of cell growth, inhibition of apoptosis, protein glycosylation, generation of MHC-associated SIMP-derived peptides). In this approach, the biological activity of SIMP or of a cell expressing SIMP (e.g. lymphocytes or a cancer cell) in the presence of a candidate compound is compared to the biological activity in its absence, under equivalent conditions. Again, the screen may begin with a pool of candidate compounds, from which one or more useful modulator compounds are isolated in a step-wise fashion. The SIMP or cell biological activity may be measured by any suitable standard assay.

The effect of candidate molecules on SIMP-biological activity may, instead, be measured at the level of translation by using the general approach described above with standard protein detection techniques, such as Western blotting or immunoprecipitation with a SIMP-specific antibody (for example, the SIMP antibody described herein).

Another method for detecting compounds that modulate the activity of SIMPs is to screen for compounds that interact physically with a given SIMP polypeptide. Depending on the nature of the compounds to be tested, the binding interaction may be measured using methods such as enzyme-linked immunosorbent assays (ELISA), filter binding assays, FRET assays, scintillation proximity assays, microscopic visualization, immunostaining of the cells, in situ hybridization, PCR, etc.

A molecule that promotes an increase in SIMP expression or SIMP activity is considered particularly useful to the invention; such a molecule may be used, for example, as a therapeutic to increase cellular levels of SIMP and thereby exploit the ability of SIMP polypeptides to increase the efficacy and/or duration of a T-cell response.

A molecule that decreases SIMP activity (e.g., by decreasing SIMP gene expression or polypeptide activity) may be used to decrease cellular proliferation. This would be advantageous in the treatment of cancer, particularly hematopoietic cancers, or other cell proliferative diseases.

Molecules that are found, by the methods described above, to effectively modulate SIMP gene expression or polypeptide activity, may be tested further in animal models. If they continue to function successfully in an in vivo setting, they may be used as therapeutics to either increase the efficacy and/or duration of a T-cell response, or to inhibit tumoral cell survival.

xii) Construction of Transgenic Animal

Previous studies have shown that the B6^{dom1} (i.e. mSIMP-derived) MiHA displays several important specific features: i) it is highly immunogenic (immunodominant) for T-lymphocytes; ii) the number of MHC-associated B6^{dom1} copies per cell is higher than for any other endogenous MHC class I-associated peptides; iii) the expression of B6^{dom1} (at the level of MHC-associated peptides) is dramatically increased (128-fold) on activated T-cells relative to resting splenocytes; and iv) B6^{dom1} is an ideal target for adoptive immunotherapy of hematologic malignancies.

Study of these important features at the molecular level was hampered by the fact that the identity of gene encoding this peptide as well as the exact peptide sequence of the B6^{dom1} MiHA were unknown. Discovery that the B6^{dom1} MiHA is encoded by the SIMP gene and that the exact sequence of the B6^{dom1} MiHA is KAPDNRETL (see exemplification section) will allow for the generation of 1) transgenic mice that express the SIMP gene or SIMP mutants at various levels in one or multiple cell lineages, 2) knock-out mice in which expression of the endogenous SIMP gene is either prevented or regulated in one or multiple cell lineages.

Characterization of SIMP genes provides information that is necessary for a SIMP knockout animal model to be developed by homologous recombination. Preferably, the model is a mammalian animal, most preferably a mouse. Similarly, an animal model of SIMP overproduction may be generated by integrating one or more SIMP sequences into the genome, according to standard transgenic techniques.

Two types of transgenic mice could be generated initially: one expressing the SIMP gene ubiquitously, the other expressing SIMP selectively in T-lymphocytes. The site of expression could be determined according to the nature of the promoter gene to which the SIMP transgene will be coupled. Ubiquitous expression of SIMP would allow to identify which tissues and organs are most sensitive to SIMP overexpression. Expression in T-cells would allow to assess to which extent overexpression of SIMP would affect the level and specificity of immune responses. Because a complete “standard knockout” would probably be not viable, it would be preferable to generate conditional knockouts where the SIMP gene expression would be inhibited at a precise time and only in selected tissue or organs using previously described methods (e.g. Labrecque et al., Immunity 15, 71-82; Polic et al., Proc. Natl. Acad. Sci. U.S.A 98, 8744-8749). Knockout and transgenic mice would provide the means, in vivo, to study SIMP cellular biology (glycosylation, antigen processing, cell proliferation) and/or to screen for therapeutic compounds.

### EXAMPLES

The examples are meant to illustrate, not to limit the invention.

### Example 1

### Discovery of the Mouse Gene Encoding the B6^{dom1} MIHA

Background

B₆^{dom1} is an immunodominant ubiquitous mice MiHA (Fontaine et al., (2001). Nat. Med. 7:789-794). Although the immunogenic properties of B6^{dom1} have been characterized (Eden et al., (1999) J. Immunol. 162:4502-4510), the identity of the gene and the protein from which the B6^{dom1} peptide was derived have remained unknown until now.

Materials and Methods

Isolation of Mouse Tissue RNA

For initial isolation of cDNA encoding the putative B6^{dom1} peptide, total RNA was isolated from various tissues of C57BL/6J mice or from the congenic B10.H7^{b} mouse strain. Routinely, a piece of liver (100 mg) was placed in 1 ml of TRIZOL™, and was subsequently homogenized using a hand-held mini-Potter homogenizer. Samples were allowed to stand for 5 min. at room temperature to fully dissociate nucleoprotein complexes; 200 μl of chloroform was added and mixed vigorously, after which samples were again left at room temperature for 2 min, followed by centrifugation at 12,000 g for 15 mins at 4 C. The aqueous (upper) phase was transferred to a clean tube, 500 μl of isopropanol was added, samples were mixed and left at room temperature for 10 min, followed by centrifugation for 10 min as above. Pellets were washed in 1 ml of 75% ethanol, centrifuged at 7,500 g for 10 min at 4° C., dried briefly in the air, and then resuspended in 200 μl RNAse-free water. The OD₂₆₀ was used to determine the concentration of the RNA obtained, which was usually well in excess of 1 μg/μl when mouse liver was used.

RT-PCR Amplification of Mouse SIMP cDNA

Total RNA prepared from mouse tissues was used as a template for subsequent RT-PCR reactions. First strand cDNA synthesis was performed using standard protocols. Briefly, a poly d(T) oligo (20 pmol) was used to prime a reverse transcription reaction using 1 μg of mouse RNA and 200U of Superscript reverse transcriptase, and the reaction was allowed to proceed for one hour at 42° C. This product was then used as a template for PCR-mediated amplification of a mouse SIMP fragment (˜400 bp) using oligonucleotides specific for the mouse gene. The oligonucleotides used were 5′-GAGAGTTCCGAGTAGAC-3′ (sense strand, corresponding to mouse SIMP nucleotides 2166-2182) and 5′-GCGTTCTCTCAAGGACTGCTG-3′ (anti-sense strand, corresponding to SIMP nucleotides 2592-2572). PCR conditions were 94° C. for 3 min, followed by 30 cycles consisting of 94° C. for 30s, 60° C. for 30s and 68° C. for 3 min, with a final extension of 10 min at 68° C. The enzyme used for PCR was Pfx polymerase (Gibco).

Full length B6 and B10.H7^{b} SIMP cDNA was isolated in a similar fashion with the single exception that a SIMP 5′ end-specific oligonucleotide corresponding to nucleotides 41-59 was used with the 3′ oligonucleotide outlined above (nucleotides 2592-2572) to amplify the 2469 bp coding sequence.

DNA Sequencing

Dideoxynucleotide DNA sequencing was performed using both manual and automated systems. For manual routine sequencing of small PCR products, we used the Redivue ³³P-ddNTP Terminator Cycle sequencing kit (Amerhsam Pharmacia Biotech), using the PCR-mediated protocol suggested by the manfacturer. For sequencing of full-length SIMP clones an automated dye terminator system was used and performed by the DNA sequencing facility at BRI. Oligonucleotides specific for mouse SIMP were chosen so as to allow reading of the entire sequence using five oligonucleotides.

Cytotoxicity Assays

Cytotoxic activity was assessed in a standard ⁵¹Cr release assay (Pion et al., 1997. Eur. J. Immunol. 27:421-430). Target blast cells, prepared by culturing C3H.SW spleen cells (3×10⁶/ml) with 5 μg/ml of Concanavalin A (Con A; Sigma Chemical Co., St-Louis, Mo.) for 48 hours, were labeled with 100 μCi Na₂⁵¹Cr (Dupont Co., Wilmington, Del.) for 90 minutes, sensitized with synthetic peptides for 90 minutes, then mixed with C3H.SW anti-C57BL/6 effector cells at a 50:1 effector to target ratio. Cells were then incubated for 4 hours at 37° C. in a humidified atmosphere of 5% CO₂. Afterwards, supernatants were harvested and counted in a gamma counter. All tests were done in triplicate. Spontaneous release was below 15%. Results are expressed as a percentage of specific lysis calculated as follows: % specific lysis=100×(experimental release−spontaneous release)/(maximum release−spontaneous release).

Results

Identification of a Candidate Gene Using Bioinformatic Tools

Elution of peptides from B6^{dom1} positive cells, HPLC separation and T-cell mediated lysis assay were previously used to identify fractions containing peptides corresponding to mouse B6^{dom1}. These peptides were then subjected to Edman degradation for peptide sequencing, and the sequence AAPDNRETF was obtained as the best candidate for the immunodominant mouse B6^{dom1} peptide, although preliminary searches in databanks revealed that no known mouse (or human) protein contained this nonameric sequence. While we were confident that this peptide was biochemically very similar to that encoded by the mouse B6^{dom1} gene, we did not rule out the possibility that it was not 100% identical to the native peptide.

Blasts of the mouse genome which were selected for candidates that were similar but not identical to the putative B6^{dom1} peptide, revealed that one gene in particular was a strong candidate, potentially encoding B6^{dom1}. This gene (Accession no. AK018758) does not have a formal name nor assigned biological role, but contains an open reading frame of 2469 bp and encodes a protein of some 823 amino acids. The candidate peptide from this protein has the sequence KAPDNRETL, differing only at positions 1 and 9 respectively from the original candidate. Since B6^{dom1} is an H2Db-associated peptide of which positions 4, 6 and 7 appear to be critical contact residues for T-cell recognition (Perreault et al., J. Clin. Invest 98:622-628), KAPDNRETL was considered a very strong candidate given that these amino acids are conserved. It was also evident from databank analysis that this gene seems to be fairly ubiquituously expressed, which was consistent with data we had previously obtained for B6^{dom1} in mouse tissues¹⁷. Given that this gene was by far the best candidate obtained (in terms of homology with the putative AAPDNRETF sequence), we decided to further investigate its potential role as the source of the immunodominant MiHA, B6^{dom1}.

Phenotype/Genotype Correlation: Genotyping of 8 Strains of Mice (4 Positive for B6^{dom1}, 4 Negative)

A fundamental requirement for identification of the candidate gene as the one encoding B6^{dom1} was that there had to be relevant differences in the coding sequences between B6^{dom1+} and B6^{dom1-} strains of mice; more specifically, for an ideal candidate there had to be sequence divergence in or adjacent to the 27 bp region encoding KAPDNRETL, the putative B6^{dom1} nonamer.

Initially, we therefore decided to compare the sequence of this region of the candidate gene between the B6 parental strain (positive) and the B10.H7^{b} congenic strain (negative). Using mouse tissue cDNA and oligonucleotides specific for the candidate gene (designed based on the DNA sequence obtained from Genebank™), we amplified a region consisting of roughly the last 400 bp of the candidate gene, which encodes a sequence containing the nine amino acid candidate peptide. The results from this analysis were of great importance because we found that the B10.H7^{b} mice contained only two single nucleotide mutations in this 400 bp fragment: one which did not alter the amino acid sequence, and another (GAG to GAT) within the 27 bp region outlined above, which changed the sequence of the B6^{dom1} candidate peptide from KAPDNRETL to KAPDNRDTL. This was very strong evidence that the candidate gene indeed coded for B6^{dom1}, not least because this amino acid change was found at position 7 in the peptide, and this position is very important for contact with the TCR¹⁵. This result made it critical to examine other mouse strains to see whether the E to D mutation was a characteristic of the other B6^{dom1}-negative strains, which would further support the contention that KAPDNRETL was indeed the native B6^{dom1} sequence, encoded by our candidate gene.

The B6, B10, LP, and 129 strains are all positive for B6^{dom1}, while the A.BY, B10.H7^{b}, C3H.SW, and BALB.B strains are negative¹⁶. Summarized in the table below are the results of the sequence analysis of the candidate peptide as encoded by the cDNA from the various strains. Of note, the fact that a mouse strain is said to be B6^{dom1}-negative, does not mean that the AK018758 gene is not expressed but rather that the sequence of its AK018758 gene is different from that of B6^{dom1}-positive mice (it does not code for the exact nonapeptide sequence recognized by B6^{dom1}-specific T-cells but rather codes for an allelic product).

TABLE 1
Genotype/phenotype comparisons

| STRAIN | B6^{DOM1} | SEQUENCE |
|---|---|---|
| B6 | + | KAPDNRETL |
| B10 | + | KAPDNRETL |
| LP | + | KAPDNRETL |
| 129 | + | KAPDNRETL |
| A.BY | − | KAPDNRDTL |
| B10.H7^{b} | − | KAPDNRDTL |
| BALB.B | − | KAPDNRDTL |
| C3H.SW | − | KAPDNRDTL |

These data were totally supportive of the hypothesis that the AK018758 gene was indeed the gene encoding the B6^{dom1} MiHA because (a) in each case only one mutation encoding an amino acid substitution was observed between strains in the 400 bp region amplified by PCR, and (b) this mutation was identical in nature and position in each B6^{dom1}-negative strain i.e. GAG to GAT (E to D). In all cases B6^{dom1} positive strains were identical to the parental B6 strain. Collectively these data are consistent with the hypothesis that we have identified (and subsequently cloned) the gene encoding mouse B6^{dom1}. At this point we decided to compare the biological activity of the wild-type and mutant peptides to determine whether the peptides KAPDNRETL and KAPDNRDTL were targets for B6^{dom1}-specific T-cell receptor-mediated recognition and cell lysis.

Recognition of the KAPDNRETL and KAPDNRDTL Peptides by B6^{dom1}-Specific CTLs

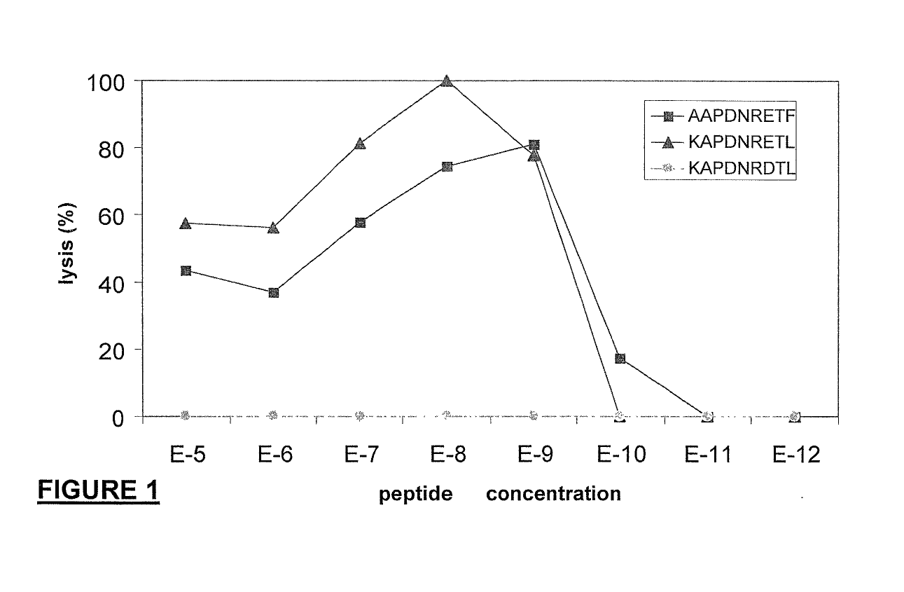

In order to prove that the KAPDNRETL peptide was the epitope recognised by B6^{dom1}-specific T-cells, we tested whether anti-B6^{dom1} T-cells (from C3H.SW mice immunised with B6 cells) would kill C3H.SW target cells coated with each of the following synthetic peptides: AAPDNRETF (previously shown to be similar to the B6^{dom1} peptide because it was recognised by B6^{dom1}-specific T-cells), KAPDNRETL (the peptide now presumed to be the natural B6^{dom1} epitope expressed in B6^{dom}1+ mice) and KAPDNRDTL (the product of the putative B6^{dom1} allele found in B6^{dom1-} strains of mice). Strikingly, the KAPDNRETL peptide was recognised more efficiently than the AAPDNRETF peptide at a 10⁻⁸ M concentration while the KAPDNRDTL peptide was not recognised even at a 10⁻⁵ M concentration (FIG. 1). Altogether, these results show that KAPDNRETL represents the real natural peptide recognised by B6^{dom1}-specific T-cells, that it is encoded by the AK01 8758 gene, and that following a single nucleotide substitution the sequence found in B6^{dom1-} mice, corresponds to KAPDNRDTL. Since i) AK018758 encodes B6^{dom1} and ii) we found that a human homolog comprises numerous peptide sequences that possess a high affinity binding motif for HLA class I molecules (see example 2), the gene encoding mouse B6^{dom1} was renamed mouse “SIMP”, that is a Source of Immunodominant MHC-associated Peptides.

### Example 2

### Discovery of the Human SIMP

Background

Given that the SIMP protein and peptides derived therefrom seemed to represent an ideal target for adoptive cancer immunotherapy, we proceeded to the identification of the human homolog of SIMP.

Materials and Methods

Isolation of Full Length Human SIMP by RT-PCR

Human SIMP cDNA was isolated by RT-PCR using human total cDNA as template (generated in an identical fashion to mouse cDNA, as described above). The oligonucleotides used for PCR were 5′-GCGGAGGACGA GCGAGACC-3′ (sense) and 5′-CGGTTCTCACMGGACMCTGC-3′ (anti-sense) to amplify the 2478 bp coding sequence (826 amino acids). PCR products were obtained from cDNAs isolated from several donors and individually sequenced to confirm the human SIMP gene sequence.

Results

Although the human genome has been sequenced, a full length human equivalent of mouse SIMP has not been identified or cloned. Blasts of the human genome nevertheless suggested that there was a human SIMP homolog. One sequence is referred to as “(moderately) similar to oligosaccharyltransferase STT3 subunit”, and corresponds to the last 286 amino acids of mouse SIMP (Accession no AK027789). Also, GenomeScan™ analysis (a new feature available in the human genome databank) of the human genome indicates that AK027789 is located on chromosome 3. Thus, the existence of a human SIMP homolog is suggested by i) the existence of a human sequence whose putative protein products would be similar to the C-terminal part of the mouse SIMP protein and ii) the fact that this sequence was mapped to human chromosome 3, a region that corresponds to the telomeric end of mouse chromosome 9 (the region encoding the B6doml MiHA, and thus, where the mouse SIMP gene is located).

Based upon available DNA sequence, we designed an oligo specific for the 3′ end of the human sequence and used this with an oligo that was specific for the 5′ end of the mouse sequence in RT-PCR experiments using human RNA. We were successful in amplifying a roughly 2,500 bp fragment containing the entire coding sequence of human SIMP: this sequence is identified in the sequence listing section as SEQ ID NO:1 and the protein product encoded by this gene is identified as SEQ ID NO:2. The initiating Met codon (ATG) and termination stop codons (TAA) are shown, at the beginning and the end of the sequence respectively.

Discussion

We have previously shown that adoptive T-cell immunotherapy targeted to B6^{dom1}, a peptide encoded by the mouse SIMP gene, could eradicate cancer cells without causing GVHD. Based on the work reported herein, we have identified the mouse B6^{dom1} gene (mSIMP), cloned its human homolog (hSIMP), and discovered that the product of the human gene contains peptide sequences with a high affinity binding motif for HLA molecules. Interestingly, the yeast analog of the mouse and human SIMP gene, STT3, is essential for cell proliferation. We intend to evaluate whether expression of human SIMP gene is required for cancer cell proliferation. The logical assumption that this is also the case for cancer cells (that is, they need to express the SIMP gene to proliferate) has important mechanistic implications because this provides a sound basis for the remarkable efficacy of SIMP-targeted immunotherapy. Accordingly, cancer cells cannot downregulate expression of this gene to evade T-cells targeted to products of the SIMP gene because SIMP expression is essential for their proliferation.

Having identified SIMP-encoded peptides with a high affinity binding motif for HLA molecules, we propose to use these peptides as targets for cancer immunotherapy. Selection of the most appropriate peptides will be based on two parameters: i) the level of expression of these peptides on various types of cancer cells (breast, prostate, lung, kidney, skin, lympho-hematopoietic tissues etc); ii) whether these peptides are polymorphic or not. Polymorphic peptides (MiHAs) will be targeted with T-cells expressing self-MHC-restricted TCR whereas non polymorphic peptides will be targeted with T-cells expressing allo-MHC TCR. Targeting can be achieved by injection of alloreactive donor T-cells or by injection of recipient T-cells transfected with the genes encoding an alloreactive TCR (derived from a human or an animal donor).

While several embodiments of the invention have been described, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention or the limits of the appended claims.

What is claimed is:

1. An isolated or purified human nucleic acid molecule encoding a human protein that is expressed ubiquitously in human cells, wherein said protein has the potential of generating a plurality of protein fragments binding with high affinity to a human HLA molecule.

2. The nucleic acid of claim 1, wherein said human protein is overexpressed in proliferative cells.

3. The nucleic acid of claim 2, wherein said proliferative cells are tumoral cells and wherein expression of said protein is essential for the tumoral cell's survival.

4. The nucleic acid of claim 1, wherein said human protein is a functional or structural homolog of yeast STT3 (SEQ ID NO: 6).

5. The nucleic acid of claim 1, wherein said human protein is a paralog of human ITM1 (SEQ ID NO: 12).

6. The nucleic acid of claim 1, comprising a polynucleotide having a nucleotide sequence coding an amino acid sequence selected from the group consisting of:
a) an amino acid sequence having greater than 71% amino acid sequence identity to SEQ ID NO:8;
b) an amino acid sequence having greater than 71% amino acid sequence identity to an amino acid sequence encoded by an open reading frame having SEQ ID NO:7;
c) an amino acid sequence having greater than 82% amino acid sequence homology to SEQ ID NO: 8;
d) an amino acid sequence having greater than 82% amino acid sequence homology to an amino acid sequence encoded by an open reading frame having SEQ ID NO: 7;
e) an amino acid sequence having greater than 97% amino acid sequence identity to SEQ ID NO: 2;
f) an amino acid sequence having greater than 97% amino acid sequence identity to an amino acid sequence encoded by an open reading frame having SEQ ID NO: 1;
g) an amino acid sequence having greater than 97% amino acid sequence homology to SEQ ID NO: 2; and
h) an amino acid sequence having greater than 97% amino acid sequence homology to an amino acid sequence encoded by an open reading frame having SEQ ID NO: 1.

7. The nucleic acid of claim 6, comprising a polynucleotide having a nucleotide sequence coding an amino acid sequence selected from the group consisting of:
a) an amino acid sequence 100% identical to SEQ ID NO: 2; and
b) an amino acid sequence 100% identical to an amino acid sequence encoded by an open reading frame having SEQ ID NO: 1.

8. The nucleic acid of claim 1, comprising a polynucleotide having a nucleotide sequence selected from the group consisting of:
a) a nucleotide sequence having greater than 63% nucleotide sequence identity with SEQ ID NO:7;
b) a nucleotide sequence having greater than 63% nucleotide sequence identity with a nucleic acid encoding an amino acid sequence of SEQ ID NO:8
c) a nucleotide sequence having at least 91% nucleotide sequence identity with SEQ ID NO: 1; and
d) a nucleotide sequence having at least 91% nucleotide sequence identity with a nucleic acid encoding an amino acid sequence of SEQ ID NO: 2.

9. The nucleic acid of claim 8, comprising a polynucleotide 100% identical to identical to SEQ ID NO: 1.

10. The nucleic acid of claim 1, wherein said HLA molecule is selected from the group consisting of HLA molecules listed in Table 1.

11. An isolated or purified human nucleic acid molecule comprising a polynucleotide having a nucleotide sequence selected from the group consisting of:
a) a nucleotide sequence having greater than 63% nucleotide sequence identity with SEQ ID NO: 7;
b) a nucleotide sequence having greater than 63% nucleotide sequence identity with a nucleic acid encoding an amino acid sequence of SEQ ID NO:8;
c) a nucleotide sequence having at least 91% nucleotide sequence identity with SEQ ID NO: 1;
d) a nucleotide sequence having at least 91% nucleotide sequence identity with a nucleic acid encoding an amino acid sequence of SEQ ID NO: 2; and
e) a nucleotide sequence complementary to any of the nucleotide sequences in (a), (b), (c) or (d).

12. The nucleic acid molecule of claim 11, wherein it comprises a polynucleotide having a nucleotide sequence selected from the group consisting of:
a) a nucleotide sequence having at least 91% nucleotide sequence identity with SEQ ID NO: 1;
b) a nucleotide sequence having at least 91% nucleotide sequence identity with a nucleic acid encoding an amino acid sequence of SEQ ID NO: 2; and
c) a nucleotide sequence complementary to any of the nucleotide sequences in (a), or (b).

13. The nucleic acid of claim 12, comprising a polynucleotide selected from the group consisting of:
a) a polynucleotide having a nucleotide sequence 100% identical to SEQ ID NO: 1;
b) a polynucleotide having a nucleotide sequence complementary to SEQ ID NO: 1;
c) a polynucleotide having at least 15 nucleotides of the polynucleotide of (a) or (b).

14. An isolated or purified nucleic acid molecule which hybridizes under high stringency conditions to any of the nucleic acid molecules of claim 13.

15. An isolated or purified human nucleic acid molecule comprising a polynucleotide having the SEQ ID NO: 1, or degenerate variants thereof, and encoding a human SIMP polypeptide.

16. The nucleic acid of claim 15, encoding the amino acid sequence of SEQ ID NO: 2 or a fragment thereof.

17. The nucleic acid of claim 15, wherein said nucleic acid is cDNA.

18. An isolated or purified human protein that is expressed ubiquitously in human cells, wherein said protein has the potential of generating a plurality of protein fragments binding with high affinity to a human HLA molecule.

19. The protein of claim 18, wherein said human protein is overexpressed in proliferative cells.

20. The protein of claim 19, wherein said proliferative cells are tumoral cells and wherein expression of said protein is essential for the tumoral cell's survival.

21. The protein of claim 18, wherein said human protein is a functional or a structural homolog of yeast STT3 (SEQ ID NO:8).

22. The protein of claim 18, wherein said human protein is a paralog of human ITM1 (SEQ ID NO: 12).

23. The protein of claim 18, wherein said fragments are selected from those comprising at least eight sequential amino acids of SEQ ID NO: 2.

24. The protein of claim 18, wherein said fragments are selected from the group consisting of the peptides listed in Table 1.

25. The protein of claim 18, wherein said HLA molecule is selected from the group consisting of HLA molecules listed in Table 1.

26. The protein of claim 18, wherein it comprises an amino acid sequence selected from the group consisting of:
a) an amino acid sequence having greater than 71% amino acid sequence identity to SEQ ID NO: 8;
b) an amino acid sequence having greater than 71% amino acid sequence identity to an amino acid sequence encoded by an open reading frame having SEQ ID NO: 7;
c) an amino acid sequence having greater than 82% amino acid sequence homology to SEQ ID NO: 8;
d) an amino acid sequence having greater than 82% amino acid sequence homology to an amino acid sequence encoded by an open reading frame having SEQ ID NO: 7;
e) an amino acid sequence having greater than 97% amino acid sequence identity to SEQ ID NO: 2;
f) an amino acid sequence having greater than 97% amino acid sequence identity to an amino acid sequence encoded by an open reading frame having SEQ ID NO: 1;
g) an amino acid sequence having greater than 97% amino acid sequence homology to SEQ ID NO: 2; and
h) an amino acid sequence having greater than 97% amino acid sequence homology to an amino acid sequence encoded by an open reading frame having SEQ ID NO: 1.

27. The protein of claim 18, wherein it comprises an amino acid sequence selected from the group consisting of:
a) an amino acid sequence 100% identical to SEQ ID NO: 2; and
b) an amino acid sequence 100% identical to an amino acid sequence encoded by an open reading frame having SEQ ID NO: 1.

28. An isolated or purified polypeptide comprising an amino acid sequence selected from the group consisting of:
a) an amino acid sequence having greater than 71% amino acid sequence identity to SEQ ID NO: 8;
b) an amino acid sequence having greater than 71% amino acid sequence identity to an amino acid sequence encoded by an open reading frame having SEQ ID NO: 7;
c) an amino acid sequence having greater than 82% amino acid sequence homology to SEQ ID NO: 8;
d) an amino acid sequence having greater than 82% amino acid sequence homology to an amino acid sequence encoded by an open reading frame having SEQ ID NO: 7;
e) an amino acid sequence having greater than 97% amino acid sequence identity to SEQ ID NO: 2;
f) an amino acid sequence having greater than 97% amino acid sequence identity to an amino acid sequence encoded by an open reading frame having SEQ ID NO: 1;
g) an amino acid sequence having greater than 97% amino acid sequence homology to SEQ ID NO: 2; and
h) an amino acid sequence having greater than 97% amino acid sequence homology to an amino acid sequence encoded by an open reading frame having SEQ ID NO: 1

29. The polypeptide of claim 28, wherein it comprises an amino acid sequence selected from the group consisting of:
a) an amino acid sequence 100% identical to SEQ ID NO: 2;
b) an amino acid sequence 100% identical to an amino acid sequence encoded by an open reading frame having SEQ ID NO: 1; and
c) an amino acid sequence consisting of at least eight consecutive amino acids of (a) or (b).

30. The polypeptide of claim 29, wherein it has the potential of generating a plurality of protein fragments binding with high affinity to a human HLA molecule.

31. A substantially pure human SIMP polypeptide, or fragment thereof.

32. The polypeptide or fragment of claim 31, wherein it comprises an amino acid sequence having greater than 97% amino acid sequence homology with a polypeptide selected from the group consisting of:
a) a polypeptide having SEQ ID NO: 2;
b) a polypeptide having an amino acid sequence encoded by an open reading frame having SEQ ID NO: 1; and
c) a polypeptide that is a fragment of (a) or (b).

33. The polypeptide or fragment of claim 32, wherein said amino acid sequence identity is about 100%.

34. A substantially pure human polypeptide that is encoded by the nucleic acid of claim 1.

35. An isolated or purified human protein that is a paralog of a human protein having SEQ ID NO:12.

36. The human protein of claim 35, wherein it comprises an amino acid sequence having at least 25% identity or at least 25% homology with SEQ ID NO:12.

37. The human protein of claim 36, wherein said percentage of identity and homology are of at least 50% respectively.

38. The protein of claim 37, wherein said percentage of identity and homology are about 56% and 59% respectively.

39. An isolated or purified polypeptide fragment, said fragment comprising at least eight sequential amino acids of SEQ ID NO: 2.

40. An isolated or purified polypeptide having a high binding affinity for a human HLA molecule, said polypeptide comprising at least eight amino acids having a sequence identity that is greater than 97% to a portion of a human protein that is expressed ubiquitously in human cells.

41. The polypeptide of claim 40, wherein said human protein is overexpressed in proliferative cells.

42. The polypeptide of claim 41, wherein said proliferative cells are tumoral cells and wherein expression of said protein is essential for the tumoral cell's survival.

43. The polypeptide of claim 40, wherein said human protein is a functional or structural homolog of yeast STT3 (SEQ ID NO: 6).

44. The nucleic acid of claim 40, wherein said human protein is a paralog of human ITM1 (SEQ ID NO: 12).

45. The polypeptide of claim 40, wherein it comprises at least eight sequential amino acids of SEQ ID NO: 2.

46. The polypeptide of claim 40, wherein it comprises an amino acid sequence encoded by a nucleotide sequence comprising at least 24 sequential nucleic acid of SEQ ID NO: 1.

47. The polypeptide of claim 40, wherein it is selected from the group consisting of the peptides listed in Table 1.

48. An antisense nucleic acid which hybridizes under high stringency condition to SEQ ID NO: 1 or to a complementary sequence thereof.

49. An antisense nucleic acid that reduces human SIMP′ cellular levels of expression.

50. The antisense of claim 49, wherein said antisense hybridizes under high stringency conditions to a genomic sequence or to a mRNA.

51. The antisense of claim 49, wherein said antisense is complementary to a nucleic acid sequence encoding a protein having SEQ ID NO: 2 or a fragment thereof.

52. A pharmaceutical composition comprising a human SIMP antisense nucleic acid.

53. A method for eliminating tumoral cells in a mammal, comprising the step of injecting, into said mammal's circulatory system, T-lymphocytes that recognize a immune complex that is present at the surface of said tumoral cells, said immune complex consisting of a SIMP protein fragment or a ITM1 protein fragment bound to an MHC molecule.

54. The method of claim 53, wherein said mammal is a human.

55. The method of claim 54, wherein immune complex consists of a hSIMP protein fragment bound to a HLA molecule, and wherein said hSIMP protein fragment comprises at least eight sequential amino acids of SEQ ID NO: 2.

56. The method of claim 55, wherein said HSIMP protein fragment is selected from the group consisting of the peptides listed in Table 1.

57. The method of claim 53, wherein said ITM1 protein fragment comprises at least eight sequential amino acids of SEQ ID NO: 12.

58. A method for increasing cell proliferation in a mammal, comprising the step of: i) contacting said cell with a SIMP polypeptide; and/or ii) increasing cellular expression levels of a SIMP polypeptide.

59. A method for modulating tumoral cell survival or for eliminating a tumoral cell in a mammal, comprising the step of reducing cellular expression levels of a SIMP polypeptide.

60. The method of claim 59, wherein said mammal is human, the method comprising the step of the step of delivering a human SIMP antisense into the tumoral cell.

61. A method for modulating an immune response in a mammal, comprising increasing in lymphoid cells of said mammals the cellular expression levels of a SIMP polypeptide.

62. The method of claim 61, for increasing the level and/or the duration of an antigen-primed lymphocyte proliferation.

63. The method of claim 61, comprising transfecting lymphocytes with a cDNA coding for a SIMP polypeptide.

64. The method of claim 61, wherein said mammal is human.

65. A method for decreasing lymphoid cells proliferation, comprising decreasing in said cells cellular expression levels of a SIMP polypeptide.

66. The method of claim 65, for suppressing an immune response responsible for an autoimmune disease or a transplant rejection.

67. The method of claim 65, comprising delivering a SIMP antisense into said lymphoid cells.

68. A nucleotide probe comprising a sequence of at least 15 sequential nucleotides of SEQ ID NO: 1 or of a sequence complementary to SEQ ID NO: 1.

69. A substantially pure nucleic acid that hybridizes to a probe of at least 40 nucleotides in length, said probe derived from SEQ ID NO:1, wherein said nucleic acid hybridizes to said probe under high stringency conditions.

70. A purified antibody that specifically binds to a purified mammalian SIMP polypeptide.

71. The antibody of claim 70, wherein the mammalian SIMP polypeptide is a human SIMP polypeptide.

72. The antibody of claim 70, wherein it binds to a polypeptide having an amino acid sequence selected from the group consisting of SEQ ID NO: 2 and SEQ ID NO: 4.

73. A monoclonal or polyclonal antibody which recognizes the human SIMP polypeptide, or fragment thereof as claimed in claim 31.

74. A method for determining the amount of a SIMP polypeptide in a biological sample, comprising the step of contacting said sample with the antibody of claim 70 or with a probe according to claim 68.

75. A method of diagnosis of a cancer in a human subject comprising the step of determining the amount of a human SIMP polypeptide in a cell or a biological sample from said subject, wherein said amount is indicative of a probability for said subject of harboring proliferating tumoral cells.

76. The method of 75, wherein said proliferating tumoral cells grow rapidly and display a short doubling time.

77. The method of 75, wherein said cancer is selected from the group consisting of: lung cancers, intestine cancers, sarcomas, prostate cancer, testis cancer, breast cancer, melanomas, pancreatic cancer and hematologic cancers.

78. A kit for determining the amount of a SIMP polypeptide in a sample, said kit comprising the antibody of claim 70 and or a probe according to claim 68, and at least one element selected from the group consisting of instructions for using said kit, reaction buffer(s), and enzyme(s).

79. A transformed or transfected cell that contains the nucleic acid of claim 1.

80. A transgenic animal generated from the cell of claim 79, wherein said nucleic is expressed in said transgenic animal.

81. A cloning or expression vector comprising the nucleic acid of claim 1.

82. The vector of claim 81, wherein said vector is capable of directing expression of the peptide encoded by said nucleic acid in a vector-containing cell.

83. A method for producing a human SIMP polypeptide comprising:
providing a cell transformed with a nucleic acid sequence encoding a human SIMP polypeptide positioned for expression in said cell;
culturing said transformed cell under conditions suitable for expressing said nucleic acid; and
producing said hSIMP polypeptide.
